(19) United States
(12) Patent Application Publication
(10) Pub. No.: US 2015/0051192 A1
(43) Pub. Date: Feb. 19, 2015

(54) BLOCKING OF CUE-INDUCED DRUG REINSTATEMENT

(71) Applicant: ALBANY MEDICAL COLLEGE, Albany, NY (US)

(72) Inventors: Stanley D. Glick, Delmar, NY (US); James E. Polston, Drexel Hill, PA (US)

(21) Appl. No.: 14/387,339

(22) PCT Filed: Mar. 25, 2013
(86) PCT No.: PCT/US13/33703

§ 371 Date: Sep. 23, 2014

Related U.S. Application Data
(60) Provisional application No. 61/615,953, filed on Mar. 27, 2012.

(51) Int. Cl. A61K 31/55; A61K 9/00

(52) U.S. Cl. CPC A61K 31/55; A61K 9/0053
USPC 514/214.02

(57) ABSTRACT

A method of preventing drug use relapse by administering an effective amount of an α3β4 nicotinic antagonist to a mammal after an initial period of drug use, and preventing a relapse of drug use. A method of preventing drug use relapse due to cue inducement by administering an effective amount of an α3β4 nicotinic antagonist to a mammal after an initial period of drug use, and preventing a relapse of drug use during cue inducement. A method of preventing drug use relapse due to cue inducement by modulating the dopaminergic mesolimbic pathway by blocking α3β4 nicotinic receptors in the habenulo-interpeduncular pathway and the basolateral amygdala of a mammal after an initial period of drug use, and preventing a relapse of drug use during cue inducement. A method of preventing drug use relapse by preventing a relapse of drug use during cue inducement.

### GRANT INFORMATION

Research in this application was supported in part by a grant from the National Institute on Drug Abuse (Grant No.: R01 DA016283). The government has certain rights in the invention.

### BACKGROUND OF THE INVENTION

1. Technical Field

The present invention relates to treatments for substance abuse. More specifically, the present invention relates to treatments for substance abuse and blocking of cue-induced drug reinstatement.

2. Background Art

Drug and alcohol misuse and abuse are leading causes of death, disability and disease in the United States today. In 2010, an estimated 131.3 million Americans were current alcohol drinkers, included 58.6 million binge drinkers and 16.9 million heavy drinkers. An estimated 69.6 million Americans reported current use of a tobacco product and an estimated 22.6 million Americans were current illicit drug users (Substance Abuse and Mental Health Services Administration, 2011). In addition, current misuse and abuse of prescription medications (including opioids and benzodiazepines) is epidemic, doubling in the last decade and now responsible for more deaths than motor vehicle accidents (Centers for Disease Control, 2011).

The financial cost to society is staggering. This includes the cost of treating drug and alcohol abuse, the cost of secondary illnesses and injuries, and all the lost earnings and years of life due to abusers' illness, incarcerations, and premature death. Other costs to society include those attributable to criminal justice, social welfare, motor vehicle accidents and fires. It was estimated that the economic cost of illicit drug abuse alone, which increased steadily in the 90's, was in excess of $160 billion for the year 2000 (Office of National Drug Control Policy, 2001). Overall, the cost of drug and alcohol abuse to US society is close to a half trillion dollars each year.

A variety of new compounds are being developed as potential treatments for drug addiction, including dopamine agonists and antagonists, GABA agonists, glutamate antagonists, monoamine oxidase B inhibitors, and opioid partial agonists. Most of these treatments are targeted at a specific drug or drug class.

The need for new pharmacological approaches to treating Substance-Related Disorders (SRDs) has never been more apparent. This is particularly true for cocaine related disorders where there is currently no approved medication. A safe, effective, orally available, low cost medication is needed. Knowledge of the neuroanatomical and neurochemical mechanisms involved in SRDs provides a rational basis for discovering critically needed new pharmacotherapies.

For more than 25 years, the dopaminergic mesolimbic system has been the major focus of research regarding mechanisms of action of drugs of abuse; however, new treatments based on this research have been slow to develop and new approaches are needed. Since the 1980's it has been known that another pathway, referred to as the dorsal diencephalic conduction system (Sutherland, 1982), functions as a reward system separate from the mesolimbic pathway in the medial forebrain bundle. This other system consists of the habenula, its afferents in the stria medullaris, and its projections via the habenulo-interpeduncular pathway in the fasciculus retroflexus to the interpeduncular nucleus.

As originally described by Herkenham and Nauta (1977), most of the afferents to the habenular nuclei course through the stria medullaris. The major inputs to the medial habenula are from the septal area and use acetylcholine, glutamate and ATP as neurotransmitters (Robertson and Edwards, 1998). Other inputs to the medial habenula include a projection from the nucleus accumbens, a GABAergic projection from the nucleus of the diagonal band (Contestabile and Fonnum, 1983) and a noradrenergic projection from the central gray area. The medial habenula also receives minor serotonergic inputs from the medial raphe nucleus via the fasciculus retroflexus. The major input to the lateral habenula comes from the entopeduncular nucleus (medial globus pallidus) and is in part GABAergic and somatostatin-containing (Ellison, 1994). Other inputs include those from the nucleus accumbens and frontal cortex; dopaminergic inputs from both the ventral tegmental area and the substantia nigra have also been described (Skagerberg et al., 1984) as have serotonergic inputs from the raphe and noradrenergic inputs from the central gray.

While the outputs of both nuclei travel in the fasciculus retroflexus, the medial habenula has its efferents in the core of the fasciculus retroflexus and projects principally to the interpeduncular nucleus, but also to the ventral tegmental area, substantia nigra and raphe nuclei. These fibers are cholinergic, glutamatergic as well as substance P-containing (Ellison, 1994). The lateral habenula, with its efferents in the mantle of the fasciculus retroflexus, has projections that are more widespread, including connections to the raphe nuclei, the ventral tegmental area, the substantia nigra, the central gray, the mediodorsal thalamus, and the lateral hypothalamus. There are connections between the two habenular nuclei (Iwahori, 1977; Cuello et al., 1978; Sutherland, 1982). In addition many of the projections of these two nuclei have extensive interconnections. The interpeduncular nucleus receives major cholinergic inputs from the medial habenula and the septal areas and projects to the raphe nuclei, the central gray and, to a lesser extent, the mediodorsal thalamus (Groenewegen et al., 1986).

The medial habenula and the interpeduncular nucleus are among the brain areas with the highest densities of nicotinic receptors (Perry and Kellar, 1995), especially α3β4 nicotinic receptors (Quick et al., 1999; Klink et al., 2001), and GABA(B) receptors (Margeta-Mitrovic et al., 1999). In addition, the medial habenula lacks NMDA glutamate receptors, having only AMPA glutamate receptors (Robertson et al., 1999).

The dorsal diencephalic conduction system, like the medial forebrain bundle, connects the limbic forebrain and the midbrain.

The results of lesion studies from the 1980's suggested that the output of the dorsal diencephalic conduction system inhibits dopaminergic activity. There are several avenues by which functional interactions can occur between the dopamine containing mesocorticolimbic pathways and the dorsal diencephalic conduction system. For example, the habenula sends input to the ventral tegmental area, and the nucleus accumbens sends input to the habenula. The interpeduncular nucleus sends input to the raphe nuclei which in turn provide input to the ventral tegmental area. And the interpeduncular nucleus sends input to the medial dorsal thalamic nucleus which projects to the prefrontal cortex, which in turn has connections to the nucleus accumbens and ventral tegmental area.

A plethora of studies has determined that drugs of abuse interact with the mesolimbic system. Less studied have been the effects of drugs of abuse on the dorsal diencephalic conduction system. Several studies, assessing local glucose utilization or expression of c-fos, have, however, clearly demonstrated that drugs of abuse, after acute and chronic administration or during withdrawal, affect the habenulo-interpeduncular system (Martin et al., 1997; Wooten et al., 1982; Kimes et al., 1990; Porrino et al., 1988; Wilkerson and London, 1989; Engber et al., 1998; Brown et al., 1992; Pang et al., 1993; Seppa et al., 2001; Grunwald et al., 1988). Opioids may interact with μ-opioid receptors that exist in high densities in the habenula (Moriwaki et al., 1996). Stimulants may interact with dopamine uptake sites located on the dopaminergic projections to the lateral habenula from the ventral tegmental area or substantia nigra. Nicotine may interact with abundant nicotinic receptors, especially the α3β4 subtype, present in the medial habenula and interpeduncular nucleus. In addition, very large and sustained doses of some of these drugs have been proven to be toxic to the habenulo-interpeduncular system (e.g., Ellison, 1992, 1994; Carlson et al., 2000, 2001; Meshul et al., 1998). Even though toxicity occurs with doses much larger than those used by human addicts, it has been suggested that “alterations in this tract would be predicted to be especially important for the genesis of the symptomatology that develops during drug binges, residual effects of such binges, and the processes underlying relapse” (Carlson et al., 2000). In fact one study using a sensitization regimen of amphetamine administration revealed that the only region where c-fos expression was enhanced was the lateral habenula (Hamamura and Ichimaru, 1997).

In summary, the dorsal diencephalic conduction system functions as a reward pathway independent from the medial forebrain bundle, although a mutual inhibitory relationship seems to exist between the two systems. The dorsal diencephalic conduction system has many connections with the dopaminergic mesolimbic system, and drugs of abuse activate both systems.

The results of many studies (e.g., Conroy et al., 1995; Lena et al., 1999) support the existence of nicotinic α3β4 receptors, composed of only the α3 and β4 subunits or, in some cases, with the addition of an α5 subunit. One important characteristic of these receptors is that they are less susceptible to desensitization than any other known nicotinic receptor subtypes and, therefore, they may still play a functional role well after other nicotinic receptors are inactivated. However, it was shown that the addition of a α5 subunit to the α3β4 receptor, while having little effect on the binding affinities for nicotinic agonists, increases the rate of desensitization and Ca++ permeability (Gerzanich et al., 1998). Using autoradiography, these receptors have been located in many brain regions. However, only a few regions (habenulo-interpeduncular system, some medullary nuclei and the pineal gland) have high densities of α3β4-like receptors; several other regions (e.g., basolateral amygdala, locus ceruleus, dorsal tegmentum, subiculum and anteroventral thalamic nucleus) show moderate densities (Perry et al., 2002; Zoli et al., 1998). The mRNA distribution of the α3 and β4 subunits as well as the corresponding proteins correlate fairly well with the autoradiographic findings (Dineley-Miler and Patrick, 1992; Le Novere et al., 1996; Yeh et al., 2001). Overall these studies clearly demonstrate that α3β4 receptors, while present in various brain regions, are more prevalent in the habenulo-interpeduncular pathway than in most other brain areas.

18-Methoxycoronaridine (18MC) is an α3β4 nicotinic antagonist that has been proposed as a treatment for addiction to a number of substances. It has been shown to reduce nicotine, cocaine, morphine, methamphetamine, and ethanol self-administration (Glick et al, 1996; Glick et al, 2000a; Maisonneuve and Glick, 1999; Rezvani et al, 1997) in rats. It has also been shown to block acquisition of a cocaine conditioned place preference (McCallum and Glick, 2009). 18-MC's primary mechanism of action appears to be through selective blockade of α3β4 nicotinic receptors (Glick et al., 2002; Pace et al., 2004). The mechanism of action of nearly every abused drug appears to involve the dopaminergic mesolimbic system; although 18-MC affects the mesolimbic dopamine (DA) system, it does so in an indirect way via other pathways (Maisonneuve and Glick, 2003). In the brain, α3β4 nicotinic receptors are preferentially localized in the medial habenula and interpeduncular nucleus, while lower densities of these receptors reside in the ventral tegmental area (Klink et al., 2001; Quick et al., 1999) and other brain regions such as the dorsolateral tegmentum and basolateral amygdala (Perry et al., 2002; Zhu et al., 2005). This could explain how 18-MC, unlike any other drug, might be used to treat multiple types of addictive disorders (e.g., opioids, stimulants, alcohol, smoking).

Substantiating the above hypothesis, 18-MC was locally administered into the medial habenula of study animals; this treatment decreased morphine, methamphetamine, and nicotine self-administration in animal models (Glick et al, 2006 and 2008). Similar results also occurred when the same treatment was locally administered (bilaterally) into the interpeduncular nucleus. These results indicated that the habenulo-interpeduncular pathway plays a critical role in modulating drug self-administration, and the results also provided direct evidence of the postulated mechanism of action of 18-MC. Importantly, a dosage (10 μg) of 18-MC that was effective when administered into the interpeduncular nucleus had no effect when administered into the ventral tegmental area—this indication of selectivity is particularly significant in that it rules out the possibility that, when injected into the interpeduncular nucleus, 18-MC might have diffused to the ventral tegmental area to produce its effect.

The dopaminergic mesolimbic system and specifically dopamine release in the nucleus accumbens have been implicated in the reinforcing actions of drugs of abuse and in craving for drugs of abuse. Based on the evidence that 18-MC acts in the habenulo-interpeduncular system to modulate drug self-administration, we investigated whether 18-MC could down regulate dopamine release in the nucleus accumbens. First, the effects of systemic 18-MC (40 mg/kg) pretreatment (19 hours beforehand) of study animals on the acute and sensitized dopamine responses to morphine and cocaine in the nucleus accumbens were examined. 18-MC pretreatment abolished the sensitized dopamine responses to both morphine and cocaine in chronic dosing models (Szumlinski et al., 2000a, 2000b; see FIGS. 12 and 13). These results were further reinforced by demonstrating that local administration of 18-MC into both the medial habenula and the interpeduncular nucleus produced similar results, strongly supporting the hypothesis that 18-MC acts in the habenulo-interpeduncular pathway to dampen the mesolimbic pathway (Taraschenko et al., 2007). These results indicate that 18-MC can reverse the sensitized dopaminergic responses to both opioids and stimulants; and this is important because dopamine sensitization is believed to be the neurochemical substrate for drug craving.

Relapse to drug usage following abstinence is a significant obstacle in the treatment of drug abuse and addiction (Koob, 2000; See, 2002). A distinctive characteristic of craving and drug seeking is that it can be induced and perpetuated by conditioned stimuli (CS) associated with drugs, even following extended periods of abstinence. Studies of CS exposure paired with drug reward have revealed that these cues are able to elicit craving and cause drug seeking behavior in both human and animal models of relapse (Childress et al, 1999; Di Ciano and Everitt, 2003; Fuchs et al, 2008; O'Brien et al, 1998). These drug-paired cues acquire increased salience through repeated association with the rewarding effects of a drug, producing conditioned reinforcement that is not easily diminished (Lee et al, 2006; Weiss et al, 2001). Therefore, it is clearly important to elucidate the behavioral and neurochemical mechanisms through which drug-associated stimuli exert their effects.

The animal reinstatement model of relapse has become a popular assay to investigate the impact that cues have on drug seeking behavior (Shaham et al, 2003). It is a powerful model to study drug craving, and the results of these studies conclusively show that discriminative, discrete, and contextual cues all have the ability to reinstate drug-seeking behavior (Atkins et al, 2008; Bossert et al, 2005; Crombag et al, 2008; Crombag and Shaham, 2002; Fuchs et al, 2007; Gabriele and See, 2010). However, the vast majority of these studies have used either simple discriminative or discrete cues (e.g., tone or light), or contextual cues (e.g., color, floor texture, bedding) that fail to replicate the complexity of environmental triggers that are likely to be present during human drug experiences.

Recent investigations show that music can serve as an effective contextual CS in rats. For instance, music has been shown to enhance MDMA-conditioned reward in rats (Feduccia and Duvauchelle, 2008); this study revealed increases in both locomotor activity and extracellular dopamine in the nucleus accumbens (NAc) after music was paired with MDMA during operant self-administration. Another recent investigation established that rats have the ability to differentiate music composed by Bach versus Stravinsky, and even transfer this ability to novel musical selections by the same composers (Otsuka et al, 2009). Furthermore, recent clinical studies have indicated that music can be used as an effective treatment for a variety of disorders. Music therapy has shown promise as an efficacious treatment for sleep disorders, anxiety, chronic stress, pain, psychosis, autism, depression, post-traumatic stress disorder, respiratory disease, and importantly, as an adjunct therapy for addiction (Bauldoff, 2009; Bradt and Dileo, 2009; de Niet et al, 2009; Gold et al, 2009; Jung and Newton, 2009; Nilsson, 2008; Rossignol, 2009). Considering the enormous potential that music therapy offers, there is an increasing need to develop preclinical models that utilize music as a significant variable. With this aim in mind, our laboratory has recently shown that after repeated pairings between music and methamphetamine, music alone can produce significant increases in locomotor activity and extracellular dopamine release in both the basolateral amygdala (BLA) and NAc in rats (Polston et al, 2011b). In a subsequent study we showed that rats demonstrate preferences between musical selections by Miles Davis and Beethoven, and that these preferences can be altered after cocaine-paired conditioning (Polston and Glick, 2011a). Taken together, these reports indicate that music can serve as an effective contextual CS in rats. However, at this time, music has yet to be used as a contextual CS in an animal reinstatement model of relapse.

One neural site shown to be crucial for cue-induced drug seeking is the BLA. The BLA is a key limbic-related region within the brain that projects heavily to the NAc, another region consistently implicated in addiction. Inactivation of the BLA through lesion or drug blockade results in attenuation of cue-induced drug seeking behaviors (Feltenstein and See, 2007; Fuchs and See, 2002). Additionally, significant increases in dopaminergic neurotransmission have been detected in the BLA after cue-induced classical conditioning procedures (Hori et al, 1993; Polston et al, 2011b). Adaptations of the cortico-limbic-striatal circuitry that take place during subjective human drug experiences may influence associative learning mediated by the BLA, the brain area thought to be ultimately responsible for cue-induced reinstatement of drug-seeking behavior (McLaughlin and Floresco, 2007).

There remains a need for a treatment that can address cue-induced drug seeking in individuals to prevent relapse of drug use. Since it is orally available and low cost to synthesize, 18-MC HCl is potentially an ideal medication to treat addictive disorders.

### SUMMARY OF THE INVENTION

The present invention provides for a method of preventing drug use relapse by administering an effective amount of an α3β4 nicotinic antagonist to a mammal after an initial period of drug use, and preventing a relapse of drug use.

The present invention also provides for a method of preventing drug use relapse due to cue inducement by administering an effective amount of an α3β4 nicotinic antagonist to a mammal after an initial period of drug use, and preventing a relapse of drug use during cue inducement.

The present invention provides for a method of preventing drug use relapse due to cue inducement by modulating the dopaminergic mesolimbic pathway by blocking α3β4 nicotinic receptors in the habenulo-interpeduncular pathway and the basolateral amygdala of a mammal after an initial period of drug use, and preventing a relapse of drug use during cue inducement.

The present invention provides for a method of preventing drug use relapse by preventing a relapse of drug use during cue inducement.

### DESCRIPTION OF THE DRAWINGS

Other advantages of the present invention are readily appreciated as the same becomes better understood by reference to the following detailed description when considered in connection with the accompanying drawings wherein:

FIG. 1 is a bar graph showing effects of music conditioning on active lever responding during daily cocaine self-administration sessions, extinction, and the reinstatement test session;

FIGS. 2A-2B are graphs showing effects of music conditioning on locomotor activity (2A) and spatial preferences within the apparatus (2B);

FIG. 3A is a graph showing the time course of extracellular dopamine during microdialysis testing on the reinstatement test day as a percentage of baseline and FIG. 3B is a depiction of representative probe placements for the basolateral amygdala;

FIG. 4 is a graph showing the effects of 18-MC on musical cue-induced reinstatement; and

FIGS. 5A and 5B are graphs showing the effects of 18-MC on locomotor activity (5A) and spatial preferences within the apparatus (5B).

### DETAILED DESCRIPTION OF THE INVENTION

Most generally, the present invention provides for methods of preventing drug relapse, especially during cue inducement. More specifically, the present invention provides for a method of preventing drug use relapse by administering an effective amount of an α3β4 nicotinic antagonist to a mammal, preferably a human, after an initial period of drug use, and preventing a relapse of drug use.

The α3β4 nicotinic antagonist can be any compound that is able to effectively block α3β4 nicotinic receptors. Preferably, the α3β4 nicotinic antagonist is a coronaridine congener (also referred to as ibogamine congeners), described in U.S. Pat. No. 6,211,360 to Glick, et al.

The coronaridine congeners are described in Formula (I) as:

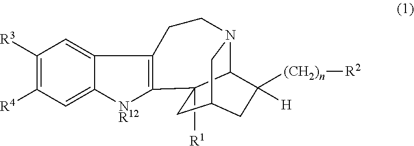

wherein n is from 0 to 8; R¹ is CH₂OH, CH(OH)R⁵, CH₂OR⁵, CO₂R⁵, C(O)NH₂, C(I)NHR⁵, C(O)NR⁵R⁶, C(O)NHNH₂, C(O)NHNHR⁵, C(O)NHNR⁵R⁶, C(O)NR⁵NH₂, C(O)NR⁵NHR⁶, C(O)NR⁵NR⁶R⁷, C(O)NHNH(C(O)R⁵), C(O)NHNR⁵(C(O)R⁶)C(O)NR⁵NH(C(O)R⁶), C(O)NR⁵NR⁶(C(O)R⁷), CN, or C(O)R⁵; R² is H, unsubstituted or substituted alkyl, YH, YR⁸, YC(O)R⁸, C(O)YR⁸, C(O)NH₂, C(O)NHR⁸, C(O)NR⁸R⁹, NH₂, NHR⁸, NR⁸R⁹, NHC(O)R⁸, or NR⁸C(O)R⁹; R³ and R⁴ are the same or different and are selected from the group consisting of H, halogens, unsubstituted or substituted alkyl, OH, OR¹⁰, NH₂, NHR¹⁰, NR¹⁰R¹¹, NHC(O)R¹⁰, or NR¹⁰C(O)R¹¹; R⁵, R⁶, R⁷, R⁸, R⁹, R¹⁰, and R¹¹ are the same or different and are selected from the group consisting of unsubstituted alkyl and substituted alkyl and substituted alkyl; R¹² is selected from the group consisting of J, unsubstituted alkyl, and substituted alkyl; and Y is O or S; provided that when n is O, R² is selected from the group consisting of H, substituted alkyl, and unsubstituted alkyl; and pharmaceutically acceptable salts thereof.

R¹ is selected from the group consisting of an alcohol, an ether, an ester, an amide, a hydrazide, a cyanide, or a ketone. Suitable alcohols include CH₂OH and CH(OH)R⁵, suitable ethers include those having the formulae CH₂OR⁵, and suitable esters include those having the formulae CO₂R⁵. Amides can be unsubstituted, such as C(O)NH₂, monosubstituted, such as, C(O)NHR⁵, or disubstituted, such as C(O)NR⁵R⁶. Suitable hydrazides include unsubstituted hydrazides, having the formula C(O)NHNH₂, monosubstituted hydrazides, having the formulae C(O)NHNHR⁵ or C(O)NR⁵NH₂, disubstituted hydrazides, having the formulae C(O)NHNR⁵R⁶ or C(O)NHR⁵NHR⁶, or trisubstituted hydrazides, having the formulae C(O)NR⁵NR⁶R⁷. The hydrazides can also contain an amide functionality at the terminal nitrogen, such as hydrazides having the formulae C(O)NHNH(C(O)R⁵), C(O)NHNR⁵(C(O)R⁶), C(O)NR⁵NH(C(O)R⁶), or C(O)NR⁵NR⁶(C(O)R⁷). Suitable ketones are those where R¹ is C(O)R⁵.

R⁵, R⁶, and R⁷ can be either unsubstituted alkyl, such as, methyl, ethyl, n-propyl, iso-propyl, n-butyl, sec-butyl, tert-butyl, n-pentyl, sec-pentyl, and neo-pentyl, hexyl, heptyl, octyl, nonyl, decyl, dodecyl, dodecyl, and the like, or substituted with any of a number of known substituents, such as sulfo, carboxy, cyano, halogen (e.g., fluoro, chloro), hydroxy, alkenyl (e.g., allyl, 2-carboxy-allyl), alkoxy (e.g., methoxy, ethoxy), aryl (e.g., phenyl, p-sulfophenyl), aryloxy (e.g., phenyloxy), carboxylate (e.g., methoxycarbonyl, ethoxycarbonyl), acyloxy (e.g., acetyloxy), acyl (e.g., acetyl, propionyl), and others known to those skilled in the art. In addition, substituted alkyls include arylalkyls, such as 2-phenyleth-1-yl, 2-phenylprop-1-yl, benzyl, and arylalkyls bearing substituents on the aromatic ring, such as 2-(5-chlorophenyl)prop-1-yl, N-piperidino, N-pyrrolidino, and N-morpholino. Each of R⁵, R⁶, and R⁷ can be the same or different and the combination is selected primarily with consideration given to the substitution's effect on water-solubility and biological compatibility, although other factors, such as availability of starting materials and synthetic ease, may enter into the selection.

Suitable esters include ethyl ester, benzyl ester, dialkylaminoalkyl esters, and, preferably, methyl ester. Amides can be, for example, N-methylamide, N-ethylamide, N,N-dimethylamide, N,N-diethylamide, N-methyl-N-ethylamide, and peptides derived from amino acids and their esters or amides. R² can also be a hydrazide, such as N′,N′-dimethylhydrazide, N′,N″-dimethylhydrazide, or preferably, unsubstituted hydrazide.

The coronaridine skeleton can be unsubstituted at the C20 position (such as in the case of desethylcoronaridine), or it can be substituted at the C20 position with an alkyl or, preferably, a derivatized alkyl. The alkyl chain, represented in the above formula by (CH₂)ₙ, can have from zero to eight carbons, inclusive, such as methyl, ethyl, propyl, butyl, pentyl, hexyl, heptyl, and octyl, and is preferably ethyl. The alkyl chain is derivatized with R² at the terminal carbon of the alkyl chain (or, in the case where n is zero, at the C20 carbon). R² is selected from the group consisting of a hydrogen, a substituted or unsubstituted alkyl, a hydroxy, an ether, a thiol, a thioether, an amine, or an acid or thioacid derivative. In cases where n is zero, R² is preferably H or substituted or unsubstituted alkyl. Illustrative examples of suitable substituted or unsubstituted alkyls include those given for R⁵, R⁶, and R⁷, above, Suitable ethers and thioethers have the formulae OR⁸ and SR⁸, respectively. Suitable amines include unsubstituted amines (NH₂), monosubstituted amines (NHR⁸), or disubstituted amines (NR⁸R⁹). Acid or thioacid derivatives can have the formulae OC(O)R⁸, SC(O)R⁸, C(O)NH₂, C(O)SR⁸, C(O)SR⁸, C(O)NHR⁸, C(O)NR⁸R⁹, NHC(O)R⁸, or NR⁸C(O)R⁹. In each of the above, R⁸ and R⁹ can be the same or different and are selected from the group consisting of substituted or unsubstituted alkyl, examples of which are the same as those given for R⁵, R⁶, and R⁷, above. As an illustration, suitable ethers and thioethers include methoxy, ethoxy, propoxy, butoxy, pentoxy, methoxyethoxymethyl ether (OCH₂OCH₂CH₂OCH₃), methylthio, ethylthio, dimethylaminoalkoxy, and sugar acetals, such as a glucoside. Suitable amine derivatives include methylamino, ethylamino, propylamino, butylamino, pentylamino, dimethylamino, diethylamino, dipropylamino, dibutylamino, methylethylamino, methylpropylamino, methylbutylamino, ethylpropylamino, ethylbutylamino, propylbutylamino, pyrrolidino, piperidino, and morpholino. Acid or thioacid derivatives can be, for example, OC(O)CH₃, OC(O)CH₂CH₃, OC(O)(CH₂)₂CH₃, OC(O)(CH₂)₃, OC(O)(CH₂)₄CH₃, OC(O)(CH₂)₅CH₃, OC(O)(CH₂)₆CH₃, OC(O)(CH₂)₁₀CH₃, OC(O)(CH₂)₁₂CH₃, SC(O)(CH₂)₂₀CH₃, SC(O)CH₃, SC(O)CH₂CH₃, SC(O)(CH₂)₂CH₃, SC(O)(CH₂)₃CH₃, SC(O)(CH₂)₄CH₃, SC(O)(CH₂)₅CH³, SC(O)(CH₂)₆CH₃, SC(O)(CH₂)₁₀CH₃, SC(O)(CH₂)₁₂CH₃, SC(O)(CH₂)₂₀CH₃, NHC(O)CH₃, NHC(O)CH₂CH₃, NHC(O)(CH₂)₂CH₃, NHC(O)(CH₂)₃, NHC(O)(CH₂)₁₀CH₃, NHC(O)(CH₂)₁₂CH₃, NHC(O)(CH₂)₂₀CH₃, N(CH₃)C(O)CH₃, N(CH₃)C(O)CH₂CH₃, N(CH₃)C(O)(CH₂)₂CH₃, N(CH₃)C(O)(CH₂)₃, N(CH₃)C(O)(CH₂)₁₀CH₃, N(CH₃)C(O)(CH₂)₁₂CH₃, N(CH₃)C(O)(CH₂)₂₀CH₃, and esters and amides derived from amino acids and amino acid amides.

R³ and R⁴ can be the same or they can be different. Each can be selected from hydrogen, halide (such as fluoride, chloride, bromide, and iodide), alkyl, hydroxy, ether, or amine. The alkyl can be substituted or unsubstituted and is exemplified by the substituted or unsubstituted alkyls used to illustrate R⁵, R⁶, and R⁷. Suitable ethers have the formulae OR¹⁹ and suitable amines include unsubstituted amines (NH₂), monosubstituted amines (NHR¹⁰), or disubstituted amines (NR₁₀R¹¹). In each of the above. R⁸ and R⁹ can be the same or different and are selected from the group consisting of substituted or unsubstituted alkyl, examples of which are the same as those given for R⁵, R⁶, and R⁷, above. As an illustration R³, R⁴, or both R³ and R⁴ can be methoxy, ethoxy, propoxy, butoxy, pentoxy, methoxyethoxymethyl ether (OCH₂OCH₂CH₂OCH₃), methylamino, ethylamino, propylamino, butylamino, pentylamino, dimethylamino, diethylamino, dipropylamino, dibutylamino, methylethylamino, methylpropylamino, methylbutylamino, ethylpropylamino, ethylbutylamino, propylbutylamino, and arylalkyl, such as benzyl. In addition, the R³ and R⁴ substituents can be linked via an alkylene, such as methylene or ethylene to form a five- or six-membered ring, such as where R³ and R⁴, together, are —OCH₂O—, —OCH₂CH₂O—, —NHCH₂O—, —NHCH₂CH₂O—, —NHCH₂NH—, and —NHCH₂CH₂NH—, R¹² can be a hydrogen, a substituted alkyl, such as an arylalkyl, or an unsubstituted alkyl. Suitable unsubstituted and substituted alkyls include those used to exemplify R⁵, R⁶, and R⁷, above.

Illustrative examples of compounds of the present invention are as follows: 18-hydroxycoronaridine; 18-hydroxyvoacangine; 18-hydroxyconopharyngine; 16-ethoxycarbonyl-18-hydroxyibogamine; 16-ethoxycarbonyl-18-hydroxyibogaine; 16-ethoxycarbonyl-18-hydroxyibogaline; 16-hydroxymethyl-18-hydroxyibogamine; 16-hydroxymethyl-18-hydroxyibogaine; 16-hydroxymethyl-18-hydroxyibogaline; 18-methoxycoronaridine; 18-methoxyvoacangine; 18-methoxyconopharyngine; 16-ethoxycarbonyl-18-methoxyibogamine; 16-ethoxycarbonyl-18-methoxyibogaine; 16-ethoxycarbonyl-18-methoxyibogaline; 16-hydroxymethyl-18-methoxyibogamine; 16-hydroxymethyl-18-methoxyibogaine; 16-hydroxymethyl-18-methoxyibogaline; 18-benzyloxycoronaridine; 18-benzyloxyvoacangine; 18-benzyloxyconopharyngine; 16-ethoxycarbonyl-18-benzyloxyibogamine; 16-ethoxycarbonyl-18-benzyloxyibogaine; 16-ethoxycarbonyl-18-benzyloxyibogaline; 18-hydroxycoronaridine laurate; 18-hydroxyvoacangine laurate; 18-hydroxyconopharyngine laurate; 16-ethoxycarbonyl-18-hydroxyibogamine laurate; 16-ethoxycarbonyl-18-hydroxyibogaine laurate; 16-ethoxycarbonyl-18-hydroxyibogaline laurate; 18-hydroxycoronaridine acetate; 18-hydroxyvoacangine acetate; 18-hydroxyconopharyngine acetate; 16-ethoxycarbonyl-18-hydroxyibogamine acetate; 16-ethoxycarbonyl-18-hydroxyibogaine acetate; 16-ethoxycarbonyl-18-hydroxyibogaline acetate; 18-hydroxycoronaridine methoxyethoxymethyl ether; 18-hydroxyvoacangine methoxyethoxymethyl ether; 18-hydroxyconopharyngine methoxyethoxymethyl ether; 16-ethoxycarbonyl-18-hydroxyibogamine methoxyethoxymethyl ether; 16-ethoxycarbonyl-18-hydroxyibogaine methoxyethoxymethyl ether; 16-ethoxycarbonyl-18-hydroxyibogaline methoxyethoxymethyl ether; and pharmaceutically acceptable salts thereof.

Most preferably, the α3β4 nicotinic antagonist is the coronaridine congener 18-MC. As shown herein, 18-MC decreases drug self-administration by indirectly modulating the dopaminergic mesolimbic pathway via blockade of α3β4 nicotinic receptors in the habenulo-interpeduncular pathway and the basolateral amygdala. While 18-MC has been used to reduce drug use during self-administration, it is shown for the first time herein that it can also reduce and prevent a relapse of drug use after the end of self-administration (i.e. an initial period of drug use). It should be understood that any of the coronaridine congeners can have a mechanism of action similar or the same as that of 18-MC, and that they can be used in any of the methods herein instead of 18-MC.

Administration of the α3β4 nicotinic antagonist in the methods herein is preferably by intraperitoneal injection. However, any other administration method can be used as described below. The α3β4 nicotinic antagonist is administered to the mammal in a dose of 0.05 mg/kg to 200 mg/kg, preferably 0.25 mg/kg to 100 mg/kg, and most preferably 0.85 mg/kg to 50 mg/kg. The α3β4 nicotinic antagonist is administering at a time period after drug use. This can be during a rehabilitation program, immediately after drug use, or at any other suitable time before a period of potential relapse.

The drug being used by the mammal in any of the methods herein can be any drug or addictive substance such as, but not limited to, a barbiturate; an opiate, such as morphine, codeine, heroin, levorphanol, meperidine, methadone, propoxyphene, acetylmethadol (LAAM), pentazocine, butorphanol, nalbuphine, buprenorphine, dezocine, fentanyl, and combinations of these opiates; a stimulant, such as d-amphetamine, 1-amphetamine, d1-amphetamine, methamphetamine, 3,4-methylenedioxy-N-methylamphetamine (MDMA) benzphetamine, phentermine, diethylpropion, phenmetrazine, phendimetrazine, chlorphentermine, clortermine, mazindol, phenylpropanolamine, cocaine, methylphenidate, nicotine, cathinone (khat plant), and combinations of these stimulants; a depressant, such as meprobamate, chlordiazepoxide, diazepam, oxazepam, lorazepam, flurazepam, prazepam, chlorazepate, alprazolam, triazolam, temazepam, halazepam, quadazepam, midazolam, estazolam, ethanol, pentobarbital, phenobarbital, secobarbital, amobarbital, delta-9-tetrahydrocannabinol (THC), and combinations of these depressants; or combinations of these addictive substances, as well as analogs and derivatives of these agents. The individual can be addicted to one of these addictive substances or to a plurality of these addictive substances.

The present invention also more specifically provides for a method of preventing drug use relapse due to cue inducement by administering an effective amount of an α3β4 nicotinic antagonist to a mammal after an initial period of drug use, and preventing a relapse of drug use during cue inducement. Any of the α3β4 nicotinic antagonists described above can be used, and preferably 18-MC is used. The α3β4 nicotinic antagonists are especially shown to be useful in preventing a relapse of drug use when the individual receives cue inducement as shown below.

The cue can be, but is not limited to, music, drugs, drug paraphernalia, seeing others using drugs, environments where drugs were consumed, environments where drugs were supplied, arousal, anxiety, discomfort, and combinations thereof.

Preventing a relapse further includes the step of reducing conditioned place preference (CPP) of the mammal.

The present invention also provides for a method of preventing drug use relapse due to cue inducement by modulating the dopaminergic mesolimbic pathway by blocking α3β4 nicotinic receptors in the habenulo-interpeduncular pathway and the basolateral amygdala of a mammal after an initial period of drug use, and preventing a relapse of drug use during cue inducement.

This particular pathway is shown to be critical in cue-induced relapse, as detailed below in Example 1. Any of the compounds that are α3β4 nicotinic antagonists described herein can be administered in order to modulate the dopaminergic mesolimbic pathway in this method.

The present invention also generally provides for a method of preventing drug use relapse by preventing a relapse of drug use during cue inducement. This can be accomplished by administering any of the compounds described herein.

Although experiments are herein focused on treating cocaine abuse, because no treatment for cocaine abuse currently exists, an innovative aspect of the present invention is that 18-MC has the potential to treat multiple forms of SRDs.

Furthermore, in developing 18-MC for clinical use, a receptor mechanism and a neuronal pathway not yet explored in medication development are being targeted. 18-MC truly represents a “paradigm shift” in the overall approach to treating SRDs. The potential benefit is extraordinary, both in terms of lives saved and economic cost to society.

18-MC can also be used in combination with other forms of psychosocial therapy. While similar to other SRD pharmacotherapies in this respect, 18-MC can occupy a unique and innovative niche, having greater efficacy than other treatments and being particularly useful in treating polydrug SRDs.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days or weeks. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

The present study had three objectives: (1) validate the effectiveness of music as a contextual conditioned stimulus in an operant reinstatement model of relapse; (2) determine, using in vivo microdialysis, if dopaminergic changes occurred during music-induced reinstatement of drug seeking; and (3) assess the efficacy of 18-MC to abate cue-induced drug seeking behaviors. All studies were conducted using a model of self-administration, extinction and reinstatement in which rats made lever presses for cocaine in the presence or absence of a musical cue (TABLE 1). The results of the present study provide novel insight into the mechanisms underlying contextual cues and associated drug-seeking behavior, and also demonstrate the effectiveness of 18-MC as a potential treatment for relapse, even in the presence of complex contextual cues.

Materials and Methods

Animals

Naïve female Sprague-Dawley rats (Taconic Germantown, N.Y.), weighing approximately 250 g at the start of the experiments, were housed individually in a temperature and humidity controlled colony room under a standard 12:12 light/dark cycle. Food and water were provided ad libitum. Protocols were designed and implemented in accordance with the “Guide for the Care and Use of Laboratory Animals” (1996) and were approved by the Institutional Animal Care and Use Committee of Albany Medical College. Rats were given one week of acclimation prior to experimental procedures.

Drugs

Cocaine hydrochloride (˜0.4 mg/kg/infusion, Sigma-Aldrich, St. Louis, Mo.) was dissolved in 0.9% sodium chloride with a 2 mg/ml drug to saline ratio, and then brought to a neutral physiological pH before use in intravenous (i.v.) self-administration sessions. 18-MC (40 mg/kg, Obiter Research LLC. Champaign, Ill.) and saline were both administered intraperitoneally (i.p.). Animals were anesthetized with sodium pentobarbital (50 mg/kg, i.p.) for both intrajugular and microdialysis cannulation surgeries. Sodium methohexital (10 mg/kg) was used to verify catheter patency. All other reagents used in conjunction with microdialysis experiments were obtained from local suppliers and were of analytical grade.

Music

Miles Davis' “Four” (Prestige Blue Haze, 1954) was the musical track used as a contextual cue in these experiments. The Miles Davis selection was chosen because it had been used successfully in past conditioning paradigms in our laboratory (Polston et al, 2011b). This musical selection was originally chosen because it has a repetitive beat and melody, helping to make it easily recognizable and identifiable. During drug training, self-administration, and applicable test sessions, “Four” was played on a continuous loop, at a volume staying between 65 and 75 decibels. This decibel range was chosen because it had been used successfully in past investigations involving rats and music (Feduccia et al, 2008; Otsuka et al, 2009; Polston et al, 2011b).

Apparatus

Experiments were conducted in rat operant conditioning chambers (ENV-009, Med Associates, St. Albans, Vt.) located within sound attenuated boxes outfitted with acoustical foam. The operant boxes were continuously ventilated with a house fan, and equipped with two retractable levers spaced approximately 20 cm apart on the front wall, with a house light mounted on the back wall of the test chamber. Infusion pumps (PHM-100VS, Med Associates, St. Albans, Vt.) located beneath the operant test chamber were used in combination with polyethylene tubing and Instech (375/22PS) swivels for i.v. drug delivery. Stereo speakers (Orb Audio, New York, N.Y.) were mounted from the ceiling and suspended above the middle of the operant boxes. These speakers were interfaced with a stereo receiver (Sony Inc., Tokyo, Japan) that controlled the musical acoustics in the operant test chambers. Additionally, infrared digital video cameras (Clover Inc., Cerritos, Calif.) were mounted from the ceiling of the operant boxes, allowing an unobstructed view of the test chamber floor. These cameras were used in conjunction with Any-Maze™ video tracking software (Stoelting Inc., Wood Dale, Ill.) to analyze locomotor activity and the time spent in predefined spatial areas within the apparatus. By operationally defining the floor (30.5 cm×31.8 cm) of the test chamber, and dividing it into three spatial zones, the program automatically generated detailed readings of the time spent in each zone in seconds and the distance that the animal traveled in meters. We defined the “active zone” (15.25 cm×15.9 cm) of the apparatus as the area containing the active drug-paired lever and the surrounding spatial area. The “inactive zone” (15.25 cm×15.9 cm) contained the inactive lever and surrounding spatial area, and the “back zone” (15.25 cm×31.8 cm) consisted of the back half of the test chamber. By operationally dividing the test chamber in this way, our system provided an automated way to determine spatial preferences within the apparatus. Videos were also periodically recorded and analyzed to ensure that Anymaze was functioning correctly.

Self-Administration Procedure

During initial shaping of the lever press response, a modular pellet dispenser (ENV-203M, Med Associates, St. Albans, Vt.) and receptacle were added to the operant test chamber, allowing delivery of a 45 mg sucrose chocolate flavored pellet (Bio-Serv, Frenchtown, N.J.). Food-deprived rats were trained to lever press for sucrose pellets during an overnight 16 h session under a fixed-ratio 1 (FR1) schedule of reinforcement. Both retractable levers were present during training, but only one (active lever) was associated with reward delivery. Responses on the other lever (inactive lever) were recorded but did not have any programmed consequences. Active lever responses resulted in immediate delivery of a food pellet, followed by retraction of both levers for a 20 second timeout period. Following the timeout, the house light would flash for 0.5 seconds, and the levers would re-emerge from the front wall of the apparatus. Rats were considered “trained” if they successfully completed 200 active lever presses during the 16 hour session.

Experiment 1:

Once the rats had successfully learned to lever press for food, they were randomly assigned to one of three treatment groups: Music, NMCond, or NMTest (refer to TABLE 1 for a detailed account of all musical treatments). Rats were subsequently anesthetized with sodium pentobarbital (50 mg/kg) and catheters were implanted in the external jugular vein according to procedures described by Weeks (1972). Rats were given a minimum of three days recovery time before drug self-administration sessions commenced. Self-administration testing began with a 16 hour nocturnal session. Each rat's catheter was flushed with 0.05 ml of saline and immediately placed in the operant box, where the animal was tethered to the drug infusion tubing. If applicable (TABLE 1), the music was then started along with the behavioral tracking system, and the levers in the operant box were deployed, initiating the beginning of the cocaine self-administration session. An active lever-response (FR1) produced a 50 μl infusion of cocaine over the course of one second, followed by retraction of both levers for a 20 second timeout period. Following the timeout, the house light would flash for 0.5 seconds, and the levers would re-emerge from the front wall of the apparatus. Since all rats generally weighed 250±20 g, each response delivered approximately 0.4 mg/kg of cocaine during the infusion. Responses on the inactive lever resulted in no programmed consequences but were recorded. Assignment of the active lever within the operant chamber was counterbalanced among subjects. At the end of the session, rats were removed from the operant box, their catheters were flushed with heparinized saline, and they were returned to the colony room. Animals had to make a minimum of 100 active cocaine responses during the overnight training session in order to move into daily self-administration sessions.

TABLE 1
Musical conditioning assignments during cocaine training, self-
administration, extinction, and reinstatement test sessions.

| Experiment | Group | Training | Self-Administration | Extinction | Reinstatement |
|---|---|---|---|---|---|
| One | Music | Music | Music | No Music | Music |
| | NMCond | No Music | No Music | No Music | Music |
| | NMTest | Music | Music | No Music | No Music |
| Two | Microdialysis | Music | Music | No Music | Music |
| Three | 18MC | Music | Music | No Music | Music |
| | Saline | Music | Music | No Music | Music |

Daily self-administration sessions followed the same protocol outlined above for the 16 hour nocturnal sessions; that is, rats were transported to the operant boxes and allowed to self-administer cocaine, either in the presence or absence of the contextual music cue. The Music treatment group was exposed to the musical cue during cocaine conditioning sessions, and reintroduced to the music during the reinstatement test. The NMCond control group was not exposed to the music during conditioning, but did receive the musical cue during the reinstatement test. The NMTest control group received music during daily cocaine sessions, but did not receive music during the reinstatement test. The duration of each of the 15 daily sessions was 90 minutes. A FR1 schedule of reinforcement was used on days 1-12, at which time rats were subsequently moved to a FR3 schedule of reinforcement for the final three cocaine-self administration sessions and all subsequent extinction and reinstatement sessions. Following the final self-administration session, catheter patency was checked by infusing a small dose (10 mg/kg) of sodium methohexital, which would immediately render the rat ataxic if the cannula was functioning properly. Only rats whose catheters were patent on day 15 were allowed to continue to the extinction and reinstatement parts of the experiment.

Following self-administration training, rats began daily 90 minute extinction sessions for five consecutive days (days 16-20). During these sessions, no music was present for any of the three treatment groups, and responses on either the previously drug paired lever or the inactive lever resulted in no drug infusions. Additionally, animals underwent 24 days of abstinence, with housing in the colony room, prior to reinstatement testing. Following this period of extinction and abstinence, both treatment (Music) and control animals (NMCond, NMTest) were tested (day 45) to determine what effect the music-drug conditioning would have on drug seeking behaviors. This model of self-administration, extinction, abstinence, and reinstatement testing followed a previously established rat protocol of reinstatement (Kelamangalath and Wagner, 2009).

Experiment 2:

Animals in this experiment underwent the exact same treatment conditions as the animals in the Music group in Experiment 1; that is, their cocaine training, extinction/abstinence, and reinstatement music conditions were identical (TABLE 1). However, at the time of the intrajugular catheterization surgery, these animals underwent an additional stereotaxic surgery for implantation of microdialysis guide cannulae. This surgery was conducted in accordance with a previously established protocol (Maisonneuve et al, 1999). Each rat had two microdialysis guide cannulae (CMA/Microdialysis AB, Stockholm, Sweden) implanted into the basolateral amygdala (BLA). Coordinates were determined according to Paxinos and Watson (Paxinos and Watson, 1986) such that, when inserted, the dialysis probe was located in the BLA (in mm, AP=−2.2; ML=±4.6; DV=−5.0, 0° lateral angle insertion). On the afternoon prior to assessment (day 44), microdialysis probes were calibrated for DA, DOPAC, and HVA to ensure recovery higher than 15% (Glick et al, 1994). Probes were discarded if they did not meet the 15% criteria. The subjects were transiently anesthetized with 25 mg/kg of Pentothal (Hospira, INC., Lake Forest, Ill.), and then placed into our operant chambers, where microdialysis probes were inserted and connected via a custom harness and tubing to both the self-administration tether and microdialysis tubing. The subjects were monitored until the effects of anesthesia had subsided, and were provided with ad libitum food and water throughout the night.

On the day of microdialysis reinstatement testing (day 45), samples were collected in tubes containing 2 μl of 1.1 N perchloric acid solution (containing 50 mg/l Na2EDTA and 50 mg/l sodium metabisulfite). The probe was continuously perfused at a flow rate of 1 μl/min with artificial cerebrospinal fluid (146 mM NaCl, 2.7 mM KCL, 1.2 mM CaCl2, 1.0 mM MgCl2). A test sample was collected for 20 minutes from each probe for each experimental subject. Six, 20 minute baseline samples were obtained during the first 2 hours of sample collection. Immediately following baseline sample collection, the reinstatement test session commenced, and the conditioned music cue was presented; four 20 minute samples were collected during behavioral testing. The cue was removed (music turned off) at the end of the 90 minute session, and an additional five 20 minute samples were collected. The dialysate samples were transferred from collection to analysis vials for DA, DOPAC, and HVA analysis by high performance liquid chromatography with electrochemical detection (HPLC-EC). Immediately following the microdialysis reinstatement experiment, subjects were sacrificed; their brains were removed and preserved for histological confirmation of guide cannulae placements. The BLA was chosen for study because it had been previously shown to respond to musical cues after drug conditioning (Polston et al, 2011b).

Experiment 3:

Animals in this experiment underwent the exact same treatment conditions as the animals in the music group in Experiment 1; that is, their cocaine training, extinction/abstinence, and reinstatement music conditions were identical (TABLE 1). However, these animals received i.p. injections of either 18-MC (40 mg/kg) or saline 20 minutes prior to the reinstatement test session.

Histology

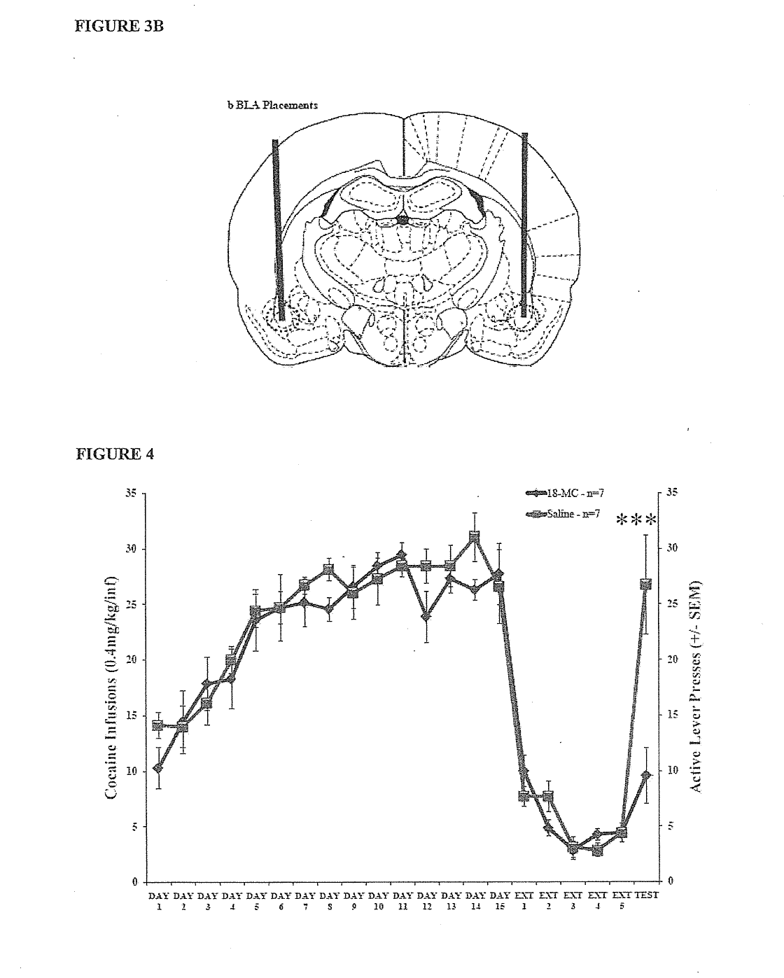

Brains were frozen at −80° C. until histology was performed utilizing a cryostat (Microm HM500M, Walldorf, Germany). Probe placements were mapped directly from the cryostat sections, and data were excluded from analysis if the probe was not located within region-specific boundaries for the BLA (refer to FIG. 3B).

HPLC

The dialysate samples were analyzed utilizing a high performance liquid chromatography system with electrochemical detection (HPLC-EC). The system consisted of an ESA 540 autosampler (ESA, North Chelmsford, Mass.), an ESA solvent delivery unit, an ESA column (MD-150/RP-C18; diameter=3.0 μm), and an ESA Coulochem II electrochemical detector with an ESA 5020 guard cell and an ESA 5014B analytical cell. The potential of the glass carbon working electrode was set at 300 mV with respect to the reference electrode. The MD-TM mobile phase (ESA, North Chelmsford, Mass.), composed of 75 mM sodium dihydrogen phosphate monohydrate, 1.7 mM 1-octanesulfonic acid sodium salt, 100 μl/l triethylamine, 25 μM EDTA in 10% acetonitrile (pH=3.0), was pumped at a flow rate of 0.530 ml/min. The electrochemical data were processed with Agilent Technologies Chem Station Plus software (Agilent Technologies, Wilmington, Del.). The software produced chromatographs, visual depictions of DA, DOPAC, and HVA concentrations (in pmol) plotted on the y-axis against the temporal representation (in minutes) for ion affinity plotted along the x-axis.

Data Analysis

All data are presented as mean±SEM. For Experiment 1, active and inactive lever presses were analyzed using a factorial Analysis of Variance (ANOVA) with Condition (Music, NMCond, or NMTest) and Trial as the independent variables. Locomotor activity data and the amount of time spent in the active, inactive, and back zones were analyzed at three time points (final day of self-administration, final day of extinction, and reinstatement test day) using one-way ANOVAs with Condition as the independent variable. All significant results were further examined by Newman-Keuls post-hoc tests.

For analysis of the microdialysis data in Experiment 2, basal levels of DA and its metabolites were expressed as pm/10 μl and were analyzed using a repeated measures ANOVA with Time as the repeated measures variable. As no significant differences were observed in the basal levels, DA and its metabolites were expressed as a percentage of the corresponding baseline means, and the percent baseline values were then used in subsequent analyses. A repeated measures ANOVA was used to evaluate differences between basal and treatment samples with Time (20 minute samples, 15 total) as the repeated measure. Significant results were further examined by Newman-Keuls post-hoc testing. To determine if animals receiving microdialysis differed in behavior prior to the reinstatement test, a factorial ANOVA was conducted on active and inactive lever presses comparing the microdialysis animals with all other groups that received the same conditioning during training, self-administration, and extinction.

In Experiment 3, active and inactive lever presses were analyzed using a factorial ANOVA with Treatment (18-MC or NaCl) and Trial as the independent variables. Locomotor activity data and the amount of time spent in the active, inactive and back zones were analyzed at three time points (final day of self-administration, final day of extinction, and reinstatement test day) using a one-way ANOVA with Treatment as the independent variable. All significant results were further examined by Newman-Keuls post-hoc tests.

Results

Experiment 1—Music-Induced Reinstatement

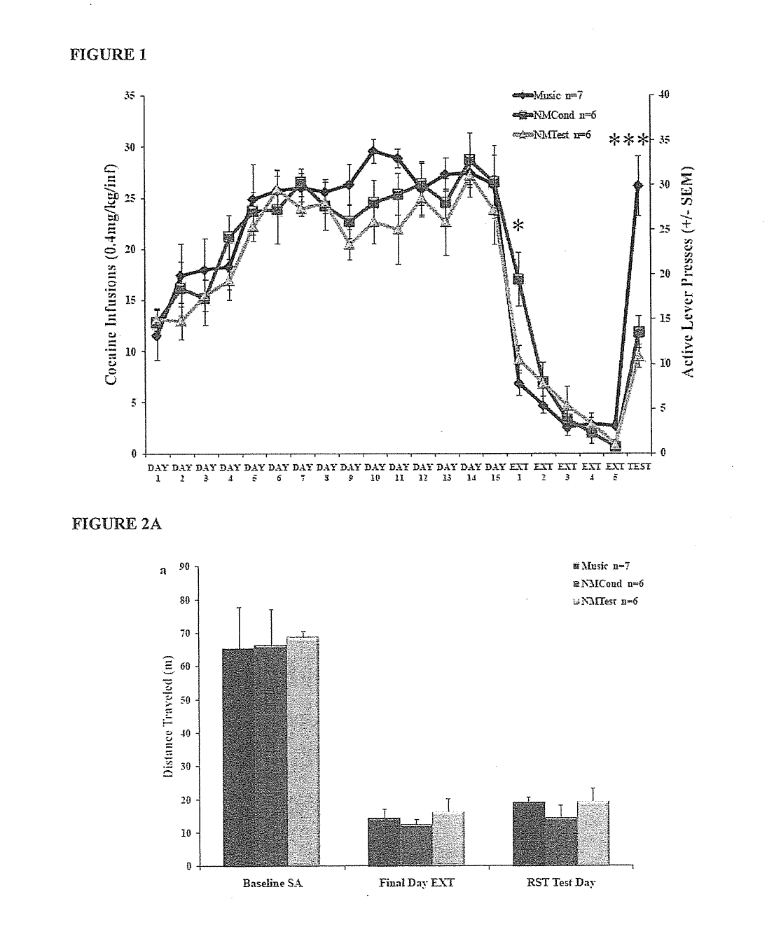

FIG. 1 depicts the average responses made for cocaine reinforcement during self-administration trials, extinction sessions, and the reinstatement test day. The factorial ANOVA revealed a significant effect of Trial (F_{(20,336)}=55.052, p<0.001) and a significant Condition× Trial interaction (F_{(40,336)}=1.643, p<0.01) but not an effect of Condition alone (F_{(2,336)}=1.369, p=0.256). Post-hoc analysis revealed that a significant difference was observed on the first day of extinction (Ext 1), where animals that had not been conditioned with music during self-administration (NMCond) made significantly more responses than animals that had been trained with music (p<0.05). Furthermore, animals in the Music condition made significantly more responses on the active lever on the reinstatement test day (Test) compared to animals in the NMCond (p<0.001) and NMTest (p<0.001) groups. There were, however, no significant differences between NMCond and NMTest groups (p=0.98) during the reinstatement test.

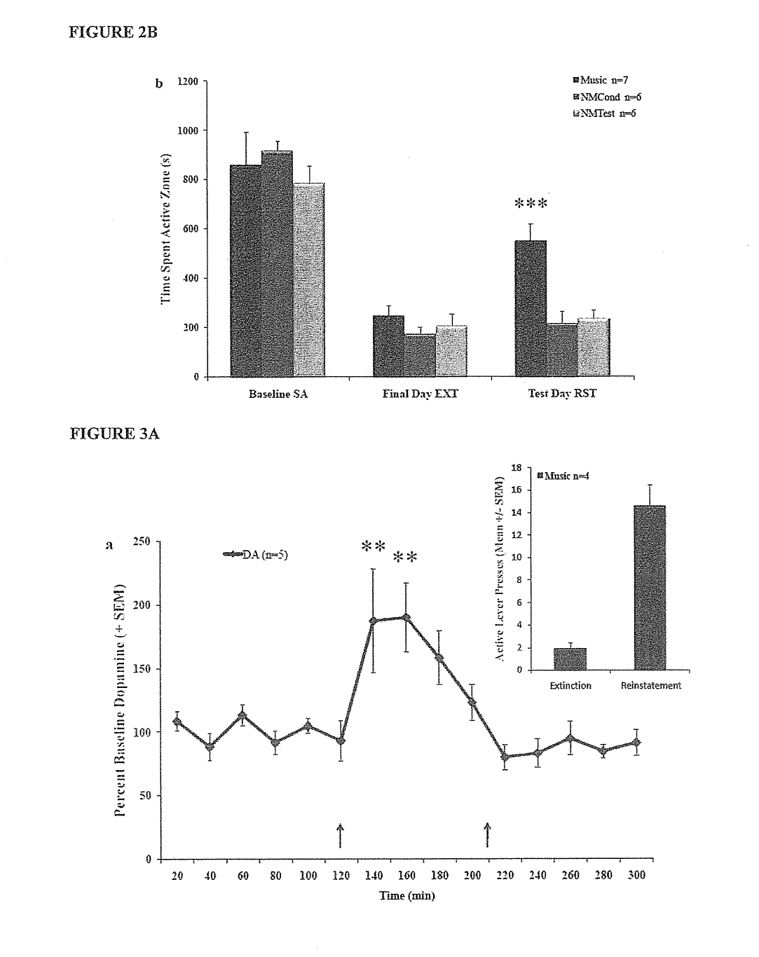

Locomotor activity data are shown in FIG. 2A. The ANOVA showed that there was no effect of Condition at any of the days tested (final day of self-administration: F_{(2,16)}=0.027, p=0.974; final day of extinction: F_{(2,16)}=0.786, p=0.472; reinstatement test day: F_{(2,16)}=0.800, p=0.466). The amount of time spent in the active zone (the area corresponding to the active lever), in the inactive zone (corresponding to the inactive lever), and in the back zone (corresponding to the remainder of the chamber) is shown in FIG. 2B. ANOVA revealed a significant effect on the reinstatement test day for the active (F_{(2,16)}=12.039, p<0.001) but not inactive (F_{(2,16)}=0.258, p=0.775) or back zones (F_{(2,16)}=1.270, p=0.308). Post-hoc analysis revealed that rats conditioned with music spent an increased amount of time in the active zone on the reinstatement day compared to NMCond (p<0.01) and NMTest (p<0.001) groups. The one-way ANOVA did not reveal any significant effects of Condition for the final day of self-administration (Active: F_{(2,16)}=0.218, p=0.807; Inactive: F_{(2,16)}=0.049, p=0.952; Back: F_{(2,16)}=0.133, p=0.876) or final day of extinction (Active: F_{(2,16)}=0.916, p=0.420; Inactive: F_{(2,16)}=0.166, p=0.849; Back: F_{(2,16)}=0.120, p=0.888).

Experiment 2—Music-Induced Dopamine Release in the BLA

TABLE 2 shows the average concentration of basal dopamine, DOPAC and HVA levels. There were no significant differences in the basal levels of dopamine (F_{(5,20)}=0.6371, p=0.674) or its metabolites (DOPAC: F_{(5,20)}=2.123, p=0.105; DOPAC: F_{(5,20)}=1.637, p=0.196). Therefore, data in FIG. 3A depicts the dopaminergic responses during the microdialysis trials as a percent of baseline. As can be seen from the graph, there was a significant efflux of dopamine (F_{(14,56)}=5.204, p<0.001) following onset of the music cue (120 min) compared to baseline (140 min: p<0.01; 160 min: p<0.01). No significant changes were observed in the levels of DOPAC (F_{(14,56)}=1.734, p=0.105) or HVA (F_{(14,56)}=1.259, p=0.262). The behavioral comparison between microdialysis animals and the other groups that received the same musical conditioning during training, self-administration and extinction sessions showed no differences in active or inactive lever presses between the groups (F_{(6,878)}=0.810, p=0.563) and no group× trial interaction (F_{(38,878)}=0.615, p=0.999). Mean (±SEM) active lever presses during the reinstatement test session were 14.6 (±1.86) and inactive lever presses during the reinstatement test session were 2.40 (±1.03).

TABLE 2
Average basal levels of extracellular DA, DOPAC, and HVA in rats
during the reinstatement test session. Mean + SEM expressed as pm/10 μl.

| Region | Neurotransmitter | Treatment | N = | Mean ± SEM |
|---|---|---|---|---|
| BLA | Dopamine | Music | 5 | 0.023 ± 0.00112 |
| | DOPAC | Music | 5 | 4.962 ± 0.12372 |
| | HVA | Music | 5 | 4.773 ± 0.08988 |

Experiment 3—18-MC Effect on Cue-Induced Reinstatement

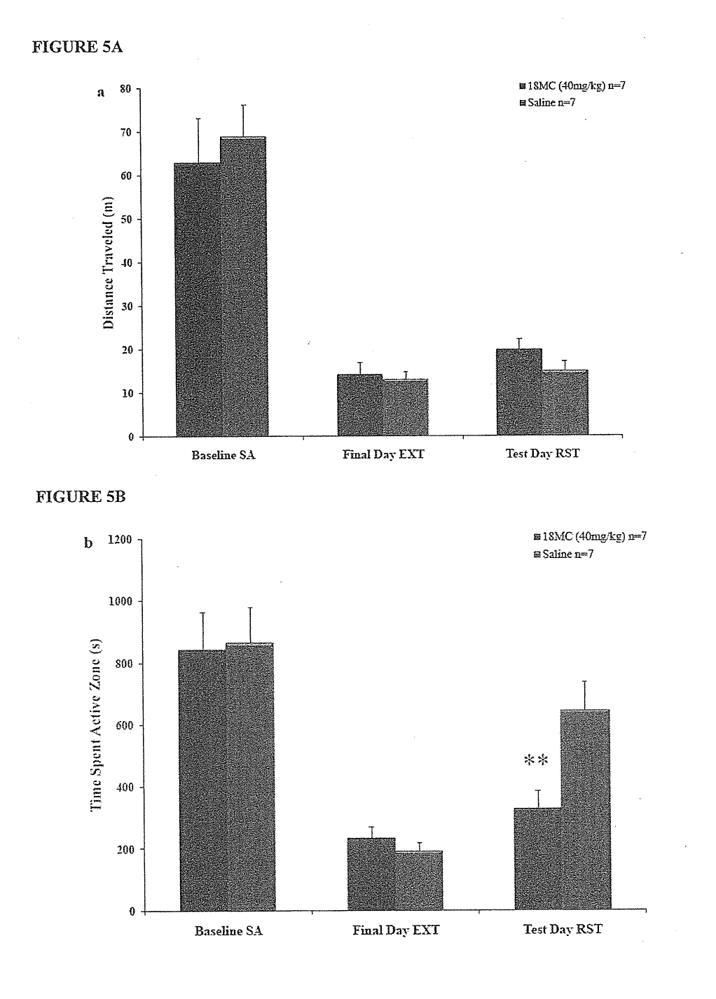

FIG. 4 depicts the average responses made for cocaine reinforcement during self-administration trials, extinction sessions, and the reinstatement test day. The factorial ANOVA revealed a significant effect of Treatment (F_{(2,251)}=3.606, p<0.05) and Trial (F_{(40,502)}=25.172, p<0.001) and a significant Treatment× Trial interaction (F_{(40,502)}=1.726, p<0.01). As can be seen from the graph, post-hoc testing showed there were no significant differences between 18-MC and saline treated groups during self-administration or extinction sessions. However, animals treated with 18-MC made significantly fewer active lever responses on the reinstatement test day (p<0.001). FIG. 5A depicts the locomotor activity of the Treatment groups, with no significant differences between groups on the final day of self-administration (F_{(1,12)}=0.235, p=0.637), the final day of extinction (F_{(1,12)}=0.141, p=0.714) or the reinstatement test day (F_{(1 12)}=2.454, p=0.143). FIG. 5B shows the average amount of time spent in the active zone during the final day of self-administration, the final day of extinction, and the reinstatement test session. ANOVA revealed no significant difference between groups in time spent in the active zone on the final day of self-administration (F_{(1,12)}=0.018, p=0.896) or the final day of extinction (F_{(1,12)}=0.900, p=0.362). There was, however, a significant decrease in the amount of time spent in the active zone for rats treated with 18-MC prior to the reinstatement test session (F_{(1 12)}=8.523, p<0.01). There was no effect of Treatment on time spent in the inactive or back zones of the operant chambers on the final day of self-administration (inactive: F_{(1 12)}=0.021, p=0.888; back: F_{(1 12})=0.001, p=0.981), the final day of extinction (inactive: F_{(1 12)}=0.007, p=0.933; back: F_{(1 12)}=0.282, p=0.605) or the reinstatement test session (inactive: F_{(1 12})=0.511, p=0.488; back: F_{(1 12)}=3.606, p=0.082).

Discussion

While the influence of conditioned cues has been extensively investigated with regard to goal directed behavior, the impact of complex environmental cues has not been comparably explored. Using the animal reinstatement model of relapse, it is shown for the first time that musical drug-paired CS have the ability to profoundly influence drug-seeking behavior following repeated pairings during cocaine self-administration. To mimic the intricate psychological processes that occur during human drug experiences, a complex contextual cue was utilized to assess associative learning processes that occur during craving. Complementing previous work (Polston et al, 2011a; Polston et al, 2011b), the present findings further support the notion that rats have the capacity to distinguish complex musical passages, and show that rats can be used in other preclinical models involving musical interventions.

The results of Experiment 1 demonstrate that animals conditioned with a musical cue (Music) show increased drug-seeking behaviors when compared to the NMCond and NMTest control groups. Music-conditioned rats made significantly more active lever responses during the reinstatement test session, indicative of increased drug craving in the presence of the musical cue (FIG. 1). These results are consistent with other cue-induced reinstatement paradigms, in which drug-paired CS have been consistently found to increase drug-seeking behavior (Crombag et al, 2002; Fuchs et al, 2008; Gabriele et al, 2010). It could be argued that the increased lever responding observed was due to chronic cocaine alone. However, both the NMCond and NMTest groups received the same cocaine reinforcement during the acquisition and maintenance phases of the experiment, and neither were significantly different from the Music group during daily self-administration and reinstatement test sessions. Differences observed between the music-conditioned animals and the control animals during the reinstatement session were most likely an effect of condition, as the music acquired increased salience during acquisition and daily cocaine sessions. When compared to the subjects that had received the musical cue during training and daily self-administration sessions, the subjects that did not receive music conditioning (NMCond) on the first day of extinction showed significantly increased active lever responding. This result is consistent with other studies showing that rats experiencing cues during self-administration extinguish more readily when those cues are removed and more readily than rats that have not had the opportunity to develop CS-drug associations (Arroyo et al, 1998; Panlilio et al, 2000). Indeed, drug-related cues produce an enduring resistance to extinction due to the associative learning that takes place during conditioned reinforcement (Weiss et al, 2001). Thus the observed differences in extinction were likely attributable to the absence of music for subjects accustomed to it during previous reinforcement sessions.

One finding that was somewhat surprising is that a locomotor effect was not found in music-conditioned animals during reinstatement test sessions (FIG. 2A). Other investigations using similar reinstatement procedures have found cue-induced locomotor activation during final testing (Feduccia et al, 2008). Moreover, it is quite common to find locomotor activation to cues that were previously associated with drug reward (Bevins et al, 2001; Rodriguez-Borrero et al, 2006). However, other studies have found no differences in locomotor activation to CS after drug-paired conditioning, and reviews show that contextual cues in particular yield mixed locomotor results (Martin-Iverson and Reimer, 1996; Tzschentke, 1998). Interestingly, closer examination of the groups' behaviors revealed that, although they did not show differences in locomotor activity, animals conditioned with the musical cue spent significantly more time in the spatial area surrounding the active lever during the reinstatement test session (FIG. 2B). This indicates that animals developed an effect analogous to a conditioned place preference (CPP) within the apparatus in the presence of the cue previously associated with cocaine reinforcement. In a typical CPP paradigm, a primary reinforcer is paired with contextual stimuli, which acquire secondary reinforcing properties. These secondary reinforcing properties, established due to classical conditioning, are capable of inducing an operant approach response or place preference. Indeed, CPP results consistently show that drug-paired environmental stimuli are capable of producing drug-seeking behavior during abstinence, indicative of drug craving (McCallum et al, 2009; Tzschentke, 2007). The fact that the animals essentially “camped out” by the previously active drug-paired lever is indicative of goal-directed behavior, and it certainly helps explain the lack of locomotor activation. An analogy to human behavior would be that an addict, after experiencing a drug-paired contextual CS, decided to “hang out” by the door of his drug distributor, rather than running aimlessly all over town.

During reinstatement sessions in Experiment 2, using in vivo microdialysis, the dopaminergic response to the cue previously associated with cocaine self-administration was examined. It was found that the presence of the musical cue elicited a substantial increase in extracellular dopamine within the BLA (FIG. 3A). Immediately following presentation of the musical cue, extracellular dopamine increases of approximately 100% were observed for the 40 minutes following cue initiation. This finding is consistent with previous work in our laboratory showing that, following repeated classical conditioning sessions with methamphetamine, music alone can increase extracellular DA in the BLA (Polston et al, 2011b). Moreover, the present microdialysis results are further corroborated by studies that have shown cue-induced increases in BLA DA in other conditioning paradigms (Suzuki et al, 2002; Yokoyama et al, 2005). These results are also consistent with the literature showing that inactivation of the BLA through lesion or drug blockade results in attenuation of cue-induced drug seeking behaviors (Feltenstein et al, 2007; Fuchs et al, 2002). Behaviorally, the animals undergoing microdialysis showed no significant differences when compared to other animals that received the same musical conditioning during training, daily self-administration sessions, and extinction. Although these rats did not make as many active lever presses during the reinstatement session, this is readily explained by the differences in protocol required for microdialysis sample collection as well as possibly by the custom harness designed for these experiments. While these harnesses were designed with intent to minimize any possible discomfort, the additional tubing and probes required for microdialysis procedures did slightly inhibit overall behavioral responding. Regardless, the animals made sufficient responses to exhibit reinstatement-like behavior, and this provided an important neurochemical measure regarding the impact of the musical cue.

While the effectiveness of the musical conditioned cue was able to be validated in Experiment 1, perhaps the most significant and important finding of this investigation is that 18-MC was able to block the cue-induced reinstatement produced by the musical CS. As can be seen in FIG. 4, 18-MC significantly attenuated responses on the previously active drug-paired lever during the reinstatement test session. While 18-MC has been shown to attenuate self-administration for multiple drugs of abuse, it has not been studied as extensively in animal models of craving (Glick et al, 1996; Glick et al, 2000a; Maisonneuve et al, 1999; Rezvani et al, 1997). One model of craving that 18-MC has been applied to is CPP, and it was shown that 18-MC was able to block the acquisition of a cocaine CPP (McCallum et al, 2009). Therefore, the fact that 18-MC was able to block cue-induced reinstatement in the form of active lever pressing was a notable finding, considering the potential it has shown for treating active drug abuse. Also of interest was the finding that 18-MC produced no changes in locomotor activity when administered prior to the reinstatement test session (FIG. 5A). These results are consistent with other of Applicants' findings showing that 18-MC produces no locomotor effects alone when compared to saline treated rats (Glick et al, 2000b). However, 18-MC was able to attenuate the music-induced CPP effect previously seen in Experiment 1. As can be seen from FIG. 5B, administration of 18-MC (40 mg/kg) prior to the reinstatement test session significantly decreased the time spent in the active zone (i.e., corresponding to the previously drug-paired lever). Thus 18-MC was able to block musical-cue induced drug seeking behaviors, both by decreasing active lever pressing and by abolishing a CPP-like effect. These effects could not be attributed to locomotor differences since 18-MC had no effect on locomotor activity. Rather, the results suggest that 18-MC's ability to attenuate drug seeking behaviors in this paradigm is due to a specific behavioral effect where subjects showed decreased interest in reinstated lever responding and decreased interest in the spatial area associated with previous drug experiences.

There are some potential mechanisms involved in this phenomenon. 18-MC appears to act in three circuits: the medial habenula-interpeduncular nucleus, basolateral amygdala-nucleus accumbens, and the dorsolateral tegmentum-ventral tegmental area. All three of these circuits appear to potentially modulate the mesolimbic dopaminergic pathway, which is the primary circuitry consistently implicated in drug addiction (Maisonneuve and Glick, 2003). However, the relative importance of these various pathways for the actions of 18-MC appear to vary with the particular reward (e.g., methamphetamine vs. sucrose; cf. Glick et al., 2008). Interestingly, the BLA, which has been shown to be critical for cue-induced reinstatement, is apparently much less important for opioid reward than for stimulant reward (Alderson et al, 2000; Olmstead and Franklin, 1997). Perhaps this helps explain why extracellular dopamine increases in the BLA have been consistently found in response to music-induced cues paired with stimulants in both a previous noncontingent drug-CS (methamphetamine) investigation (Polston et al, 2011b) and in the current investigation where drug (cocaine) was contingently administered in a reinstatement paradigm. The common factor in both of these paradigms was that the musical cue was paired with a stimulant (methamphetamine or cocaine); it would be interesting to determine if these musical cues would be as effective both behaviorally and neurochemically (i.e., within the BLA) with an opioid.

Alpha3beta4 nicotinic receptors are preferentially localized in the medial habenula and interpeduncular nucleus, with lower densities in the basolateral amygdala (Perry et al., 2002; Zhu et al., 2005), and the hypothesis is that 18-MC decreases drug self-administration by indirectly modulating the dopaminergic mesolimbic pathway via blockade of α3β4 nicotinic receptors in the habenulo-interpeduncular pathway and the basolateral amygdala. Perhaps a similar mechanism helps explain why 18-MC was so effective at blocking drug craving in our current model, as disruption of the BLA circuitry appears to be necessary to prevent cue-induced reinstatement (McLaughlin et al, 2007). 18-MC has been proposed as a treatment for addiction to multiple drugs, as well as showing promise as a treatment for obesity (Maisonneuve et al, 2003; Taraschenko et al, 2008). Antagonism of α3β4 nicotinic receptors represents a relatively novel approach to treating multiple addictive disorders, dampening the impact of the mesolimbic pathway through indirect modulation via the habenulo-interpeduncular pathway. Pleasurable music induces neurological reactions in humans that are comparable to the effects induced by drugs of abuse. For example, highly enjoyable music has been shown to activate brain regions such as the nucleus accumbens, ventral tegmental area, amygdala, and prefrontal cortex. Enhanced functional connectivity between brain regions that mediate reward can help explain why listening to music is regarded as a highly pleasurable human experience (Blood and Zatorre, 2001; Menon and Levitin, 2005). It has also been demonstrated that music increases dopaminergic neurotransmission in the brain (Sutoo and Akiyama, 2004). The fact that human beings find music rewarding can help explain why music therapy has shown such promising results across a vast spectrum of disorders. However, the goal herein was not to see if rats had an appreciation for Miles Davis, but rather to determine whether a complex musical passage could effectively be used as a contextual CS in an animal reinstatement paradigm. Studies demonstrating that music can serve as an effective contextual CS in rats are an important first step in creating preclinical models that involve music.

While the influence of simple CS on goal-directed behavior has been explored thoroughly, more complex contextual CS have not been adequately investigated. Utilization of a complex contextual musical cue allowed for examination of associative learning that may be comparable to the psychological processes that occur during subjective human drug experiences. The present study is the first to give instrumental control of cocaine to a lower order species in the presence of a complex musical cue. The results clearly showed that music can indeed serve as an effective contextual CS in rats. Most importantly, the findings demonstrated that 18-MC has the ability to block musical cue-induced reinstatement, consistent with its potential use to treat drug seeking and taking in humans.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

Acroff, K. and Sclafani, A., Sucrose-induced hyperphagia and obesity in rats fed a macronutrient self-selection diet, Physiol. Behav., 44 (1988) 181-187. (PMID3237824)
Alderson H L, Robbins T W, Everitt B J (2000). Heroin self-administration under a second-order schedule of reinforcement: acquisition and maintenance of heroin-seeking behaviour in rats. Psychopharmacology (Berl) 153(1): 120-133.
Alkondon, M. and Albuquerque, E. X., Diversity of nicotinic acetylcholine receptors in rat hippocampal neurons, I. Pharmacological and functional evidence for distinct structural subtypes. J. Pharmacol. Exp. Ther., 265 (1993) 1455-73. (PMID8510022)
Arroyo M, Markou A, Robbins T W, Everitt B J (1998). Acquisition, maintenance and reinstatement of intravenous cocaine self-administration under a second-order schedule of reinforcement in rats: effects of conditioned cues and continuous access to cocaine. Psychopharmacology (Berl) 140(3): 331-344.
Atkins A L, Mashhoon Y, Kantak K M (2008). Hippocampal regulation of contextual cue-induced reinstatement of cocaine-seeking behavior. Pharmacol Biochem Behav 90(3): 481-491.
Bauldoff G S (2009). Music: More than just a melody. Chron Respir Dis 6(4): 195-197.
Bevins R A, Besheer J, Pickett K S (2001). Nicotine-conditioned locomotor activity in rats: Dopaminergic and GABAergic influences on conditioned expression. Pharmacology Biochemistry and Behavior 68(1): 135-145.
Blander, A. and Wise, R. A., Anatomical mapping of brain stimulation reward sites in the anterior hypothalamic area: special attention to the stria medullaris, Brain Res., 483 (1989) 12-16. (PMID2706499)
Blood A J, Zatorre R J (2001). Intensely pleasurable responses to music correlate with activity in brain regions implicated in reward and emotion. Proc Natl Acad Sci USA 98(20): 11818-11823.
Bossert J M, Ghitza U E, Lu L, Epstein D H, Shaham Y (2005). Neurobiology of relapse to heroin and cocaine seeking: An update and clinical implications. European Journal of Pharmacology 526(1-3): 36-50.
Bradt J, Dileo C (2009). Music for stress and anxiety reduction in coronary heart disease patients. Cochrane Database Syst Rev(2): CD006577.
Brown, E. E., Robertson, G. S., and Fibiger, H. C., Evidence for conditional neuronal activation following exposure to a cocaine-paired environment: role of forebrain limbic structures, J. Neurosci., 12 (1992) 4112-4121. (PMID1403102)
Cappendijk, S. L. and Dzoljic, M. R., Inhibitory effects of ibogaine on cocaine self-administration in rats, Eur. J. Pharmacol., 241 (1993) 261-265. (PMID8243561)
Carlson, J., Armstrong, B., Switzer, R. C., and Ellison, G., Selective neurotoxic effects of nicotine on axons in fasciculus retroflexus further support evidence that this a weak link in brain across multiple drugs of abuse, Neuropharmacology, 39 (2000) 2792-2798. (PMID11044749)
Carlson, J., Noguchi, K., and Ellison, G., Nicotine produces selective degeneration in the medial habenula and fasciculus retroflexus, Brain Res., 906 (2001) 127-134. (PMID11430869)
Childress A R, Mozley P D, McElgin W, Fitzgerald J, Reivich M, O'Brien C P (1999). Limbic activation during cue-induced cocaine craving. American Journal of Psychiatry 156(1): 11-18.
Christoph, G. R., Leonzio, R. J., and Wilcox, K. S., Stimulation of the lateral habenula inhibits dopamine-containing neurons in the substantia nigra and ventral tegmental area of the rat, J. Neurosci., 6 (1986) 613-619. (PMID 3958786)
King, C.-H. R., Meckler, H., Herr, R. J., Trova, M. P., Glick, S. D., and Maisonneuve, I. M., Synthesis of enantiomerically pure (+)- and (−)-18-Methoxycoronaridine hydrochloride and their preliminary assessment as anti-addictive agents, Bioorg. Med. Chem. Lett., 10 (2000) 473-476. (PMID10743951)
Clarke, P. B., Hamill, G. S., Nadi, N. S., Jacobowitz, D. M., and Pert, A., 3H-nicotine- and 125I-alpha-bungarotoxin-labeled nicotinic receptors in the interpeduncular nucleus of rats, II. Effects of habenular deafferentation, J. Comp. Neurol., 251 (1986) 407-413. (PMID3771837)
Compton, W. M., Cocaine use and HIV risk in out of treatment drug abusers, Drug Alcohol Depend., 58 (2000) 215-218. (PMID10759031)
Conroy, W. G. and Berg, D. K., Neurons can maintain multiple classes of nicotinic acetylcholine receptors distinguished by different subunit compositions, J. Bio. Chem., 270 (1995) 4424-4431. (PMID7876208)
Contestabile, A. and Fonnum, F., Cholinergic and GABAergic forebrain projections to the habenula and nucleus interpeduncularis: surgical and kainic acid lesions, Brain Res., 275 (1983) 287-297. (PMID6626983)
Corrigall, W. A., Coen, K M., Zhang, J., and Adamson, L., Pharmacological manipulations of the pedunculopontine tegmental nucleus in the rat reduce self-administration of both nicotine and cocaine, Psychopharmacology, 160 (2002) 198-205. (PMID11875638)
Crombag H S, Bossert J M, Koya E, Shaham Y (2008). Review. Context-induced relapse to drug seeking: a review. Philos Trans R Soc Lond B Biol Sci 363(1507): 3233-3243.
Crombag H S, Shaham Y (2002). Renewal of drug seeking by contextual cues after prolonged extinction in rats. Behav Neurosci 116(1): 169-173.
Cuello, A. C., Emson, P. C., Paxinos, G., and Jessell, T., Substance P containing and cholinergic projections from the habenula, Brain Res., 149 (1978) 413-429. (PMID352479)
de Niet G, Tiemens B, Lendemeijer B, Hutschemaekers G (2009). Music-assisted relaxation to improve sleep quality: meta-analysis. J Adv Nurs 65(7): 1356-1364.
Di Ciano P, Everitt B J (2003). Differential control over drug-seeking behavior by drug-associated conditioned reinforcers and discriminative stimuli predictive of drug availability. Behavioral Neuroscience 117(5): 952-960.
Dineley-Miller, K. and Patrick, J., Gene transcripts for the nicotinic acetylcholine receptor subunit, beta4, are distributed in multiple areas of the rat central nervous system, Brain Res. Mol., 16 (1992) 339-344. (PMID1337943)
Ellison, G., Continuous amphetamine and cocaine have similar neurotoxic effects in lateral habenular nucleus and fasciculus retroflexus, Brain Res., 598 (1992) 353-356. (PMID1486500)
Ellison, G., Stimulant-induced psychosis, the dopamine theory of schizophrenia, and the habenula, Brain Res. Brain Res. Rev., 19 (1994) 223-239. (PMID7914793)
Engber, T. M., Koury, E. J., Dennis, S. A., Miller, M. S., Contreras, P. C., and Bhat, R. V., Differential patterns of regional c-Fos induction in the rat brain by amphetamine and the novel wakefulness-promoting agent modafinil, Neurosci. Lett., 241 (1998) 95-98. (PMID9507929)
Feduccia A A, Duvauchelle C L (2008). Auditory stimuli enhance MDMA-conditioned reward and MDMA-induced nucleus accumbens dopamine, serotonin and locomotor responses. Brain Research Bulletin 77(4): 189-196.
Feltenstein M W, See R E (2007). NMDA receptor blockade in the basolateral amygdala disrupts consolidation of stimulus-reward memory and extinction learning during reinstatement of cocaine-seeking in an animal model of relapse. Neurobiology of Learning and Memory 88(4): 435-444.
Flores, C. M., Rogers, S. W., Pabreza, L. A., Wolfe, B. B., and Kellar, K. J., A subtype of nicotinic cholinergic receptor in rat brain is composed of alpha 4 and beta 2 subunits and is up-regulated by chronic nicotine treatment, Mol. Pharmacol., 41 (1992) 31-37. (PMID1732720)
Fryer, J. D. and Lukas, R. J., Noncompetitive functional inhibition at diverse, human nicotinic acetylcholine receptor subtypes by bupropion, phencyclidine, and ibogaine, J. Pharmacol. Exp. Ther., 288 (1999) 88-92. (PMID9862757)
Fu, F., Matta, S. G., McIntosh, J. M., and Sharp, B. M., Inhibition of nicotine-induced hippocampal release in rats by alpha-conotoxins MII and AuIB microinjected into the locus coeruleus, Neurosci. Lett., 266 (1999) 113-116. (PMID10353340)
Fuchs R A, Eaddy J L, Su Z I, Bell G H (2007). Interactions of the basolateral amygdala with the dorsal hippocampus and dorsomedial prefrontal cortex regulate drug context-induced reinstatement of cocaine-seeking in rats. Eur J Neurosci 26(2): 487-498.
Fuchs R A, Ramirez D R, Bell G H (2008). Nucleus accumbens shell and core involvement in drug context-induced reinstatement of cocaine seeking in rats. Psychopharmacology 200(4): 545-556.
Fuchs R A, See R E (2002). Basolateral amygdala inactivation abolishes conditioned stimulus- and heroin-induced reinstatement of extinguished heroin-seeking behavior in rats. Psychopharmacology (Berl) 160(4): 425-433.
Gabriele A, See R E (2010). Reversible inactivation of the basolateral amygdala, but not the dorsolateral caudate putamen, attenuates consolidation of cocaine-cue associative learning in a reinstatement model of drug-seeking. Eur J Neurosci 32(6): 1024-1029.
Gerzanich, V., Wang, F., Kuryatov, A. and Lindstrom, J., Alpha 5 subunit alters desensitization, pharmacology, Ca++ permeability and Ca++ modulation of human neuronal alpha 3 nicotinic receptors, J. Pharmacol. Exp. Ther., 286 (1998) 311-320. (PMID9655874)
Glick S D, Dong N, Keller Jr R W, Carlson J N (1994). Estimating extracellular concentrations of dopamine and 3,4-dihydroxyphenylacetic acid in nucleus accumbens and striatum using microdialysis: Relationships between in vitro and in vivo recoveries. Journal of Neurochemistry 62(5): 2017-2021.
Glick, S. D., Kuehne, M. E., Raucci, J., Wilson, T. E., Larson, D., Keller, R. W., and Carlson, J. N., Effects of iboga alkaloids on morphine and cocaine self-administration in rats: relationship to tremorigenic effects and to effects on dopamine release in nucleus accumbens and striatum, Brain Res., 657 (1994) 14-22. (PMID7820611)
Glick, S. D., Kuehne, M. E., Maisonneuve, I. M., Bandarage, U. K., and Molinari, H. H., 18-Methoxycoronaridine, a non-toxic iboga alkaloid congener: effects on morphine and cocaine self-administration and on mesolimbic dopamine release in rats, Brain Res., 719 (1996) 29-35. (PMID8782860)
Glick, S. D. and Maisonneuve, I. M., Development of novel medications for drug addiction. The legacy of an African shrub, Ann. N.Y. Acad. Sci., 909 (2000a) 88-103. (PMID10911925)
Glick, S. D. and Maisonneuve, I. M., New Medications for Drug Abuse, Ann. N.Y. Acad. Sci., 909 (2000b).
Glick, S. D., Maisonneuve, I. M., Visker, K. E., Fritz, K. A., Bandarage, U. K., and Kuehne, M. E., 18-Methoxycoronardine attenuates nicotine-induced dopamine release and nicotine preferences in rats, Psychopharmacology, 139 (1998) 274-280. (PMID9784085)
Glick, S. D., Maisonneuve, I. M., Hough, L. B., Kuehne, M. E., and Bandarage, U. K., (±)-18-Methoxycoronaridine: A novel iboga alkaloid congener having potential anti-addictive efficacy, CNS Drug Review, 5 (1999) 27-42.
Glick, S. D., Maisonneuve, I. M., and Dickinson, H. A., 18-MC reduces methamphetamine and nicotine self-administration in rats, Neuroreport, 11 (2000a) 2013-2015. (PMID10884062)
Glick, S. D., Maisonneuve, I. M., and Szumlinski, K. K., 18-Methoxycoronaridine (18-MC) and ibogaine: comparison of antiaddictive efficacy, toxicity, and mechanisms of action, Ann. N.Y. Acad. Sci., 914 (2000b) 369-386. (PMID11085336)
Glick, S. D., Maisonneuve, I. M., Dickinson, H. A., and Kitchen, B. A., Comparative effects of dextromethorphan and dextrorphan on morphine, methamphetamine, and nicotine self-administration in rats, Eur. J. Pharmacol., 422 (2001) 87-90. (PMID11430918)
Glick, S. D., Maisonneuve, I. M., and Kitchen, B. A., Modulation of nicotine self-administration in rats by combination therapy with agents blocking alpha3 beta4 nicotinic receptors, Eur. J. Pharmacol., 448 (2002a) 185-191. (PMID12144940)
Glick, S. D., Maisonneuve, I. M., Kitchen, B. A., and Fleck, M. W., Antagonism of alpha3 beta4 nicotinic receptors as a strategy to reduce opioid and stimulant self-administration, Eur. J. Pharmacol., 438 (2002b) 99-105. (PMID11906717)
Glick, S. D., Ramirez, R. L., Livi, J. M. and Maisonneuve, I. M., 18-Methoxycoronaridine acts in the medial habenula and/or interpeduncular nucleus to decrease morphine self-administration in rats. Europ. J. Pharmacol., 537 (2006) 94-98. (PMID16626688)
Glick, S. D., Rossman, K., Steindorf, S., Maisonneuve, I. M., and Carlson, J. N., Effects and aftereffects of ibogaine on morphine self-administration in rats, Eur. J. Pharmacol., 195 (1991) 341-345. (PMID1868880)
Glick, S. D., Sell, E. M. and Maisonneuve, I. M., Brain regions mediating α3β4 nicotinic antagonist effects of 18-MC on methamphetamine and sucrose self-administration. Europ. J. Pharmacol., 599 (2008) 91-95. (PMID18930043)
Gold C, Solli H P, Kruger V, Lie S A (2009). Dose-response relationship in music therapy for people with serious mental disorders: systematic review and meta-analysis. Clin Psychol Rev 29(3): 193-207.
Gottesfeld, Z., Massari, V. J., Muth, E. A., and Jacobowitz, D. M., Stria medullaris: a possible pathway containing GABAergic afferents to the lateral habenula, Brain Res., 130 (1977) 184-189. (PMID884518)
Groenewegen, H. J., Ahlenius, S., Haber, S. N., Kowall, N. W., and Nauta, W. J., Cytoarchitecture, fiber connections, and some histochemical aspects of the interpeduncular nucleus in the rat, J. Comp Neurol., 249 (1986) 65-102. (PMID2426312)
Grunwald, F., Schrock, H., Theilen, H., Biber, A., and Kuschinsky, W., Local cerebral glucose utilization of the awake rat during chronic administration of nicotine, Brain Res., 456 (1988) 350-356. (PMID3208084)
Hamamura, T. and Ichimaru, Y., Amphetamine sensitization augments amphetamine-induced Fos expression in the lateral habenula, Brain Res., 767 (1997) 140-143. (PMID9365026)
Harsch, H. H., Pankiewicz, J., Bloom, A. S., Rainey, C., Cho, J. K., Sperry, L., and Stein, E. A., Hepatitis C virus infection in cocaine users—a silent epidemic, Community Ment. Health J., 36 (2000) 225-233. (PMID10933240)
Herkenham, M. and Nauta, W. J., Afferent connections of the habenular nuclei in the rat. A horseradish peroxidase study, with a note on the fiber-of-passage problem, J. Comp Neurol., 173 (1977) 123-146. (PMID845280)
Hernandez, S. C., Bertolino, M., Xiao, Y., Pringle, K. E., Caruso, F. S., and Kellar, K. J., Dextromethorphan and its metabolite dextrorphan block alpha3beta4 neuronal nicotinic receptors, J. Pharmacol. Exp. Ther., 293 (2000) 962-967. (PMID10869398)
Hirsch, E., Ball, E., and Godkin, L., Sex differences in the effects of voluntary activity on sucrose-induced obesity, Physiol. Behav., 29 (1982) 253-262. (PMID7146130)
Hori K, Tanaka J, Nomura M (1993). Effects of discrimination learning on the rat amygdala dopamine release: a microdialysis study. Brain Res 621(2): 296-300.
Iwahori, N., A Golgi study on the habenular nucleus of the cat, J. Comp Neurol., 72 (1977) 319-344. (PMID319124)
Jun, J. H. and Schindler, C. W., Dextromethorphan alters methamphetamine self-administration in the rat, Pharmacol. Biochem. Behav., 67 (2000) 405-409. (PMID11164066)
Jung X T, Newton R (2009). Cochrane Reviews of non-medication-based psychotherapeutic and other interventions for schizophrenia, psychosis, and bipolar disorder: A systematic literature review. Int J Ment Health Nurs 18(4): 239-249.
Kanarek, R. B. and Marks-Kaufman, R., Developmental aspects of sucrose-induced obesity in rats, Physiol. Behav., 23 (1979) 881-885. (PMID523544)
Kelamangalath L, Wagner J J (2009). Effects of abstinence or extinction on cocaine seeking as a function of withdrawal duration. Behav Pharmacol 20(2): 195-203.
Kimes, A. S., Bell, J. A., and London, E. D., Clonidine attenuates increased brain glucose metabolism during naloxone-precipitated morphine withdrawal, Neuroscience, 34 (1990) 633-644. (PMID2352645)
Klink, R., de Kerchove, D. A., Zoli, M., and Changeux, J. P., Molecular and physiological diversity of nicotinic acetylcholine receptors in the midbrain dopaminergic nuclei, J. Neurosci., 21 (2001) 1452-1463. (PMID11222635)
Koob G F (2000) Neurobiology of addiction. Toward the development of new therapies. In Annals of the New York Academy of Sciences pp 170-185.
Koob, G. F. and Le Moal, M., Drug addiction, dysregulation of reward, and allostasis, Neuropsychopharmacology, 24 (2001) 97-129. (PMID11120394)
Lanca, A. J., Adamson, K. L., Coen, K. M., Chow, B. L. C., and Corrigall, W. A., The pedunculopontine tegmental nucleus and the role of cholinergic neurons in nicotine self-administration in the rat: a correlative neuroanatomical and behavioral study, Neuroscience, 96 (2000) 735-742. (PMID10727791)
Lee J L C, Milton A L, Everitt B J (2006). Cue-induced cocaine seeking and relapse are reduced by disruption of drug memory reconsolidation. Journal of Neuroscience 26(22): 5881-5887.
Lena, C., Changeux, J. P., and Mulle, C., Evidence for “preterminal” nicotinic receptors on GABAergic axons in the rat interpeduncular nucleus, J. Neurosci., 13 (1993) 2680-2688. (PMID8501532)
Lena, C., de Kerchove, D. A., Cordero-Erausquin, M., Le Novere, N., del Mar Arroyo-Jimenez, M. and Changeux, J. P., Diversity and distribution of nicotinic acetylcholine receptors in the locus ceruleus neurons, Proc. Natl. Acad. Sci. U.S.A., 96 (1999) 12126-12131. (PMID10518587)
Le Novere, N., Zoli, M., and Changeux, J. P., Neuronal nicotinic receptor alpha 6 subunit mRNA is selectively concentrated in catecholaminergic nuclei of the rat brain, Eur. J. Neurosci. 8 (1996) 2428-2439. (PMID8950106)
Lisoprawski, A., Herve, D., Blanc, G., Glowinski, J., and Tassin, J. P., Selective activation of the mesocortico-frontal dopaminergic neurons induced by lesion of the habenula in the rat, Brain Res., 183 (1980) 229-234. (PMID7357405)
Maisonneuve, I. M., Keller, R. W., and Glick, S. D., Interactions between ibogaine, a potential anti-addictive agent, and morphine: an in vivo microdialysis study, Eur. J. Pharmacol., 199 (1991) 35-42. (PMID1893925)
Maisonneuve, I. M., Rossman, K. L., Keller, R. W., and Glick, S. D., Acute and prolonged effects of ibogaine on brain dopamine metabolism and morphine-induced locomotor activity in rats, Brain Res., 575 (1992) 69-73. (PMID1504783)
Maisonneuve, I. M. and Glick, S. D., Attenuation of the reinforcing efficacy of morphine by 18-methoxycoronaridine, Eur. J. Pharmacol., 383 (1999) 15-21. (PMID10556676)
Maisonneuve, I. M., Warner, L. M., and Glick, S. D., Biphasic dose-related effects of morphine on dopamine release, Drug Alcohol Depend., 65 (2001) 55-63. (PMID11714590)
Maisonneuve I M, Glick S D (2003). Anti-addictive actions of an iboga alkaloid congener: a novel mechanism for a novel treatment. Pharmacol Biochem Behav 75(3): 607-618.
Margeta-Mitrovic, M., Mitrovic, I., Riley, R. C., Jan, L. Y., and Basbaum, A. I., Immunohistochemical localization of GABA(B) receptors in the rat central nervous system, J. Comp Neurol., 405 (1999) 299-321. (PMID10076927)
Marks, M. J., Whiteaker, P., Grady, S. R., Picciotto, M. R., McIntosh, J. M. and Collins, A. C., Characterization of I¹²⁵ epibatidine binding and nicotinic agonist-mediated (86) Rb(+) efflux in interpeduncular nucleus and inferior colliculus of beta2 null mutant mice, J. Neurochem. 81 (2002) 1102-1115. (PMID12065623)
Martin, T. J., Miller, M., Jr., Dworkin, S. I., Smith, J. E., and Porrino, L. J., Alteration of local cerebral glucose utilization following intravenous administration of heroin in Fischer 344 rats, Brain Res., 755 (1997) 313-318. (PMID9175898)
Martin-Iverson M T, Reimer A R (1996). Classically conditioned motor effects do not occur with cocaine in an unbiased conditioned place preferences procedure. Behav Pharmacol 7(4): 303-314.
Matsuda, Y. and Fujimura, K., Action of habenular efferents on ventral tegmental area neurons studied in vitro, Brain Res. Bull., 28 (1992) 743-749. (PMID1617458)
McCallum S E, Glick S D (2009). 18-Methoxycoronaridine blocks acquisition but enhances reinstatement of a cocaine place preference. Neurosci Lett 458(2): 57-59.
McCulloch, J., Savaki, H. E., and Sokoloff, L., Influence of dopaminergic systems on the lateral habenular nucleus of the rat, Brain Res., 194 (1980) 117-124. (PMID7378832)
McLaughlin R J, Floresco S B (2007). The role of different subregions of the basolateral amygdala in cue-induced reinstatement and extinction of food-seeking behavior. Neuroscience 146(4): 1484-1494.
Menon V, Levitin D J (2005). The rewards of music listening: response and physiological connectivity of the mesolimbic system. Neuroimage 28(1): 175-184.
Meshul, C. K., Noguchi, K., Emre, N., and Ellison, G., Cocaine-induced changes in glutamate and GABA immunolabeling within rat habenula and nucleus accumbens, Synapse, 30 (1998) 211-220. (PMID9723791)
Miller, D. K., Wong, E. H., Chestnut, M. D., and Dwoskin, L. P., Reboxetine: functional inhibition of monoamine transporters and nicotinic acetylcholine receptors, J. Pharmacol. Exp. Ther., 302 (2002) 687-695. (PMID12130733)
Moriwaki, A., Wang, J. B., Svingos, A., van Bockstaele, E., Cheng, P., Pickel, V., and Uhl, G. R., Mu opiate receptor immunoreactivity in rat central nervous system, Neurochem. Res., 21 (1996) 1315-1331. (PMID8947922)
Mulle, C., Vidal, C., Benoit, P., and Changeux, J. P., Existence of different subtypes of nicotinic acetylcholine receptors in the rat habenulo-interpeduncular system, J. Neurosci., 11 (1991) 2588-2597. (PMID1869929)
Nilsson U (2008). The anxiety- and pain-reducing effects of music interventions: a systematic review. Aorn J 87(4): 780-807.
Nishikawa, T., Fage, D., and Scatton, B., Evidence for, and nature of, the tonic inhibitory influence of habenulointerpeduncular pathways upon cerebral dopaminergic transmission in the rat, Brain Res., 373 (1986) 324-336. (PMID2424555)
O'Brien C P, Childress A R, Ehrman R, Robbins S J (1998). Conditioning factors in drug abuse: Can they explain compulsion? Journal of Psychopharmacology 12(1): 15-22.
Olmstead M C, Franklin K B (1997). The development of a conditioned place preference to morphine: effects of lesions of various CNS sites. Behav Neurosci 111(6): 1313-1323.
Olmstead, M. C., Munn, E. M., Franklin, K. B. J., and Wise, R. A., Effects of pedunculopontine tegmental nucleus lesions on responding for intravenous heroin under different schedules of reinforcement, J. Neurosci., 18 (1998) 5035-5044. (PMID9634569)
Otsuka Y, Yanagi J, Watanabe S (2009). Discriminative and reinforcing stimulus properties of music for rats. Behavioural Processes 80(2): 121-127.
Pace, C. J., Glick, S. D., Maisonneuve, I. M., He, L. W., Jokiel, P. A., Kuehne, M. E. and Fleck, M. W., Novel iboga alkaloid congeners block nicotinic receptors and reduce drug self-administration. Europ. J. Pharmacol., 492 (2004) 159-167. (PMID15178360)
Panchal, V, Taraschenko, O. D., Maisonneuve, I. M, and Glick, S. D.—Attenuation of morphine withdrawal signs by intracerebral administration of 18-methoxycoronaridine. Europ. J. Pharmacol., 525 (2005) 98-104. (PMID16289028)
Pang, Y., Kiba, H., and Jayaraman, A., Acute nicotine injections induce c-fos mostly in non-dopaminergic neurons of the midbrain of the rat, Mol. Brain. Res., 20 (1993) 162-170. (PMID8255178)
Panlilio L V, Weiss S J, Schindler C W (2000). Effects of compounding drug-related stimuli: escalation of heroin self-administration. J Exp Anal Behav 73(2): 211-224.
Papke, R. L., Sanberg, P. R., and Shytle, R. D., Analysis of mecamylamine stereoisomers on human nicotinic receptor subtypes, J. Pharmacol. Exp. Ther., 297 (2001) 646-656. (PMID11303054)
Paxinos G, Watson C (1986). The rat brain in stereotaxic coordinates. Academic Press Inc.: San Diego, Calif.
Perry, D. C. and Kellar, K. J., [3H]epibatidine labels nicotinic receptors in rat brain: an autoradiographic study, J. Pharmacol. Exp. Ther., 275 (1995) 1030-1034. (PMID7473129)
Perry, D. C., Xiao, Y., Nguyen, H. N., Musachio, J. L., Davila-Garcia, M. I. and Kellar, K. J., Measuring nicotinic receptors with characteristics of alpha4beta2, alpha3beta2 and alpha3beta4 subtypes in rat tissues by autoradiography, J. Neurochem., 82 (2002) 468-481. (PMID12153472)
Pizzolato, G., Soncrant, T. T., and Rapoport, S. I., Haloperidol and cerebral metabolism in the conscious rat: relation to pharmacokinetics, J. Neurochem., 43 (1984) 724-732. (PMID6540296)
Polston J E, Glick S D (2011a). Music-induced context preference following cocaine conditioning in rats. Behav Neurosci 125(4): 674-680.
Porrino, L. J., Domer, F. R., Crane, A. M., and Sokoloff, L., Selective alterations in cerebral metabolism within the mesocorticolimbic dopaminergic system produced by acute cocaine administration in rats, Neuropsychopharmacology, 1 (1988) 109-118. (PMID3251493)
Quick, M. W., Ceballos, R. M., Kasten, M., McIntosh, J. M., and Lester, R. A., Alpha3beta4 subunit-containing nicotinic receptors dominate function in rat medial habenula neurons, Neuropharmacology, 38 (1999) 769-783. (PMID10465681)
Rauhut, A. S., Mullins, S. N., Dwoskin, L. P., and Bardo, M. T., Reboxetine: attenuation of intravenous nicotine self-administration in rats, J. Pharmacol. Exp. Ther., 303 (2002) 664-672. (PMID12388649)
Rezvani, A. H., Overstreet, D. H., and Lee, Y. W., Attenuation of alcohol intake by ibogaine in three strains of alcohol-preferring rats, Pharmacology, Biochem. Behav., 52 (1995) 615-620. (PMID8545483)
Rezvani, A. H., Overstreet, D. H., Yang, Y., Maisonneuve, I. M., Bandarage, U. K., Kuehne, M. E., and Glick, S. D., Attenuation of alcohol consumption by a novel nontoxic ibogaine analogue (18-methoxycoronaridine) in alcohol-preferring rats, Pharmacol. Biochem. Behav., 58 (1997) 615-619. (PMID9300627)
Rho, B. and Glick, S. D., Effects of 18-methoxycoronaridine on acute signs of morphine withdrawal in rats, Neuroreport, 9 (1998) 1283-1285. (PMID9631413)
Robertson, S. J. and Edwards, F. A., ATP and glutamate are released from separate neurones in the rat medial habenula nucleus: frequency dependence and adenosine-mediated inhibition of release, J. Physiol, 508 (1998) 691-701. (PMID9518726)
Robertson, S. J., Burnashev, N., and Edwards, F. A., Ca2+ permeability and kinetics of glutamate receptors in rat medial habenula neurones: implications for purinergic transmission in this nucleus, J. Physiol, 518 (1999) 539-549. (PMID10381598)
Robinson, T. E. and Berridge, K. C., The psychology and neurobiology of addiction: an incentive-sensitization view, Addiction, 95 Suppl 2 (2000) S91-117. (PMID11002906)
Rodríguez-Borrero E, Bernardo Colón A, Burgos-Mártir M A, Álvarez Carillo J E, Estrada del Campo Y, Abella-Ramírez C, et al (2006). NMDA antagonist AP-5 increase environmentally induced cocaine-conditioned locomotion within the nucleus accumbens. Pharmacology Biochemistry and Behavior 85(1): 178-184.
Rompre, P. P. and Miliaressis, E., Pontine and mesencephalic substrates of self-stimulation, Brain Res., 359 (1985) 246-259. (PMID4075148)
Rossignol D A (2009). Novel and emerging treatments for autism spectrum disorders: a systematic review. Ann Clin Psychiatry 21(4): 213-236.
Sasaki, K., Suda, H., Watanabe, H., and Yagi, H., Involvement of the entopeduncular nucleus and the habenula in methamphetamine-induced inhibition of dopamine neurons in the substantia nigra of rats, Brain Res. Bull., 25 (1990) 121-127. (PMID2207698)
See R E (2002). Neural substrates of conditioned-cued relapse to drug-seeking behavior. Pharmacol Biochem Behav 71(3): 517-529.
Seppa, T., Salminen, O., Moed, M., and Ahtee, L., Induction of Fos-immunostaining by nicotine and nicotinic receptor antagonists in rat brain, Neuropharmacology, 41 (2001) 486-495. (PMID11543769)
Shaham Y, Shalev U, Lu L, De Wit H, Stewart J (2003). The reinstatement model of drug relapse: history, methodology and major findings. Psychopharmacology (Berl) 168(1-2): 3-20.
Sharkey, J., McBean, D. E., and Kelly, P. A., Acute cocaine administration: effects on local cerebral blood flow and metabolic demand in the rat, Brain Res., 548 (1991) 310-314. (PMID1868341)
Skagerberg, G., Lindvall, O., and Bjorklund, A., Origin, course and termination of the mesohabenular dopamine pathway in the rat, Brain Res., 307 (1984) 99-108. (PMID6087992)
Speakman, J., Hambly, C., Mitchell, S., and Krol, E., Animal Models of Obesity, Obesity Reviews, 8 (Suppl. 1) (2007) 55-61. (PMID17316303)
Steinmiller, C. L., Maisonneuve, I. M., and Glick, S. D., Effects of dextromethorphan on dopamine release in the nucleus accumbens: interactions with morphine, Pharmacol. Biochem. Behav., 74 (2003) 803-810. (PMID12667894)
Sutherland, R. J. and Nakajima, S., Self-stimulation of the habenular complex in the rat, J. Comp Physiol Psychol., 95 (1981) 781-791. (PMID6975784)
Sutherland, R. J., The dorsal diencephalic conduction system: a review of the anatomy and functions of the habenular complex, Neurosci. Biobehav. Rev., 6 (1982) 1-13. (PMID7041014)
Sutoo D, Akiyama K (2004). Music improves dopaminergic neurotransmission: demonstration based on the effect of music on blood pressure regulation. Brain Res 1016(2): 255-262.
Suzuki T, Ishigooka J, Watanabe S, Miyaoka H (2002). Enhancement of delayed release of dopamine in the amygdala induced by conditioned fear stress in methamphetamine-sensitized rats. Eur J Pharmacol 435(1): 59-65.
Szumlinski, K. K., Maisonneuve, I. M., and Glick, S. D., The potential anti-addictive agent, 18-methoxycoronaridine, blocks the sensitized locomotor and dopamine responses produced by repeated morphine treatment, Brain Res., 864 (2000a) 13-23. (PMID10793182)
Szumlinski, K. K., McCafferty, C. A., Maisonneuve, I. M., and Glick, S. D., Interactions between 18-methoxycoronaridine (18-MC) and cocaine: dissociation of behavioural and neurochemical sensitization, Brain Res., 871 (2000b) 245-258. (PMID10899291)
Szumlinski, K. K., Balogun, M. Y., Maisonneuve, I. M., and Glick, S. D., Interactions between iboga agents and methamphetamine sensitization: studies of locomotion and stereotypy in rats, Psychopharmacology, 151 (2000c) 234-241. (PMID10972470)
Taraschenko, O. D., Panchal, V., Maisonneuve, I. M., and Glick, S. D., Is antagonism of α3β4 nicotinic receptors a strategy to reduce morphine dependence? Europ. J. Pharmacol., 513 (2005) 207-218. (PMID15862802)
Taraschenko, O. D., Rubbinaccio, N. Y., Maisonneuve, I. M., and Glick, S. D., 18-Methoxycoronaridine: a potential new treatment for obesity in rats? Psychopharmacology, 201 (2008) 339-350. (PMID18751969)
Taraschenko, O. D., Shulan, J. M., Maisonneuve, I. M. and Glick, S. D., 18-MC acts in the medial habenula and interpeduncular nucleus to attenuate dopamine sensitization to morphine in the nucleus accumbens. Synapse, 61 (2007) 547-560. (PMID17447255)
Trugman, J. M., Arnold, W. S., Touchet, N., and Wooten, G. F., D1 dopamine agonist effects assessed in vivo with [14C]-2-deoxyglucose autoradiography, J. Pharmacol. Exp. Ther., 250 (1989) 1156-1160. (PMID2674418)
Trugman, J. M., James, C. L., and Wooten, G. F., D1/D2 dopamine receptor stimulation by L-dopa. A [14C]-2-deoxyglucose autoradiographic study, Brain, 114 (1991) 1429-1440. (PMID1829645)
Tzschentke T M (1998). Measuring reward with the conditioned place preference paradigm: a comprehensive review of drug effects, recent progress and new issues. Prog Neurobiol 56(6): 613-672.
Tzschentke T M (2007). Measuring reward with the conditioned place preference (CPP) paradigm: update of the last decade. Addict Biol 12(3-4): 227-462.
Vachon, M. P. and Miliaressis, E., Dorsal diencephalic self-stimulation: a movable electrode mapping study, Behav. Neurosci., 106 (1992) 981-991. (PMID1472297)
Wechsler, L. R., Savaki, H. E., and Sokoloff, L., Effects of d- and I-amphetamine on local cerebral glucose utilization in the conscious rat, J. Neurochem., 32 (1979) 15-22. (PMID759566)
Weiss F, Martin-Fardon R, Ciccocioppo R, Kerr T M, Smith D L, Ben-Shahar O (2001). Enduring resistance to extinction of cocaine-seeking behavior induced by drug-related cues. Neuropsychopharmacology 25(3): 361-372.
Whiteaker, P., Peterson, C. G., Xu, W., McIntosh, J. M., Paylor, R., Beaudet, A. L., Collins, A. C. and Marks, M. J., Involvement of the alpha3 subunit in central nicotinic binding populations, J. Neurosci., 22 (2002) 2522-2529. (PMID11923417)
Wilkerson, G. and London, E. D., Effects of methylenedioxymethamphetamine on local cerebral glucose utilization in the rat, Neuropharmacology, 28 (1989) 1129-1138. (PMID2572994)
Wooten, G. F., DiStefano, P., and Collins, R. C., Regional cerebral glucose utilization during morphine withdrawal in the rat, Proc. Natl. Acad. Sci. U.S.A, 79 (1982) 3360-3364. (PMID6954484)
Wonnacott, S., Presynaptic nicotinic ACh receptors, TINS, 30 (1997) 92-98. (PMID9023878)
Wu, D., Otton, S. V., Kalow, W., and Sellers, E. M., Effects of route of administration on dextromethorphan pharmacokinetics and behavioral response in the rat, J. Pharmacol. Exp. Ther., 274 (1995) 1431-1437. (PMID7562518)
Yeh, J. J., Yasuda, R. P., Davila-Garcia, M. I., Xiao, Y., Ebert, S., Gupta, T., Kellar, K. J. and Wolfe, B. B., Neuronal nicotinic acetylcholine receptor alpha3 subunit protein in rat brain and sympathetic ganglion measured using a subunit-specific antibody: regional and ontogenic expression, J. Neurochem. 77 (2001) 336-346. (PMID11279289)
Yokoyama M, Suzuki E, Sato T, Maruta S, Watanabe S, Miyaoka H (2005). Amygdalic levels of dopamine and serotonin rise upon exposure to conditioned fear stress without elevation of glutamate. Neurosci Lett 379(1): 37-41.
Zhang, W., Ramamoorthy, Y., Tyndale, R. F., Glick, S. D., Maisonneuve, I. M., Kuehne, M. E. and Sellers, E. M. Metabolism of 18-methoxycoronaridine, an ibogaine analogue, to 18-hydroxycoronaridine by genetically variable CYP2C19. Drug Metab. Disp., 30 (2002) 663-669. (PMID12019193)
Zoli, M., Lena, C., Picciotto, M. R., and Changeux, J. P., Identification of four classes of brain nicotinic receptors using beta2 mutant mice, J. Neurosci., 18 (1998) 4461-4472. (PMID9614223)

What is claimed is:

1. A method of preventing drug use relapse, including the steps of:
administering an effective amount of an α3β4 nicotinic antagonist to a mammal after an initial period of drug use; and
preventing a relapse of drug use.

2. The method of claim 1, wherein the α3β4 nicotinic antagonist is a coronaridine congener.

3. The method of claim 2, wherein the coronaridine congener is chosen from the group consisting of 18-hydroxycoronaridine; 18-hydroxyvoacangine; 18-hydroxyconopharyngine; 16-ethoxycarbonyl-18-hydroxyibogamine; 16-ethoxycarbonyl-18-hydroxyibogaine; 16-ethoxycarbonyl-18-hydroxyibogaline; 16-hydroxymethyl-18-hydroxyibogamine; 16-hydroxymethyl-18-hydroxyibogaine; 16-hydroxymethyl-18-hydroxyibogaline; 18-methoxycoronaridine; 18-methoxyvoacangine; 18-methoxyconopharyngine; 16-ethoxycarbonyl-18-methoxyibogamine; 16-ethoxycarbonyl-18-methoxyibogaine; 16-ethoxycarbonyl-18-methoxyibogaline; 16-hydroxymethyl-18-methoxyibogamine; 16-hydroxymethyl-18-methoxyibogaine; 16-hydroxymethyl-18-methoxyibogaline; 18-benzyloxycoronaridine; 18-benzyloxyvoacangine; 18-benzyloxyconopharyngine; 16-ethoxycarbonyl-18-benzyloxyibogamine; 16-ethoxycarbonyl-18-benzyloxyibogaine; 16-ethoxycarbonyl-18-benzyloxyibogaline; 18-hydroxycoronaridine laurate; 18-hydroxyvoacangine laurate; 18-hydroxyconopharyngine laurate; 16-ethoxycarbonyl-18-hydroxyibogamine laurate; 16-ethoxycarbonyl-18-hydroxyibogaine laurate; 16-ethoxycarbonyl-18-hydroxyibogaline laurate; 18-hydroxycoronaridine acetate; 18-hydroxyvoacangine acetate; 18-hydroxyconopharyngine acetate; 16-ethoxycarbonyl-18-hydroxyibogamine acetate; 16-ethoxycarbonyl-18-hydroxyibogaine acetate; 16-ethoxycarbonyl-18-hydroxyibogaline acetate; 18-hydroxycoronaridine methoxyethoxymethyl ether; 18-hydroxyvoacangine methoxyethoxymethyl ether; 18-hydroxyconopharyngine methoxyethoxymethyl ether; 16-ethoxycarbonyl-18-hydroxyibogamine methoxyethoxymethyl ether; 16-ethoxycarbonyl-18-hydroxyibogaine methoxyethoxymethyl ether; 16-ethoxycarbonyl-18-hydroxyibogaline methoxyethoxymethyl ether; and pharmaceutically acceptable salts thereof.

4. The method of claim 1, further including the step of indirectly modulating the dopaminergic mesolimbic pathway via blockade of α3β4 nicotinic receptors in the habenulo-interpeduncular pathway and the basolateral amygdala.

5. The method of claim 1, wherein said administering step is further defined as administering the α3β4 nicotinic antagonist by intraperitoneal injection in a dose of 0.05 mg/kg to 200 mg/kg.

6. The method of claim 1, wherein the drug is chosen from the group consisting of a barbiturate, morphine, codeine, heroin, levorphanol, meperidine, methadone, propoxyphene, acetylmethadol (LAAM), pentazocine, butorphanol, nalbuphine, buprenorphine, dezocine, fentanyl, d-amphetamine, 1-amphetamine, d1-amphetamine, methamphetamine, 3,4-methylenedioxy-N-methylamphetamine (MDMA) benzphetamine, phentermine, diethylpropion, phenmetrazine, phendimetrazine, chlorphentermine, clortermine, mazindol, phenylpropanolamine, cocaine, methylphenidate, nicotine, cathinone (khat plant), meprobamate, chlordiazepoxide, diazepam, oxazepam, lorazepam, flurazepam, prazepam, chlorazepate, alprazolam, triazolam, temazepam, halazepam, quadazepam, midazolam, estazolam, ethanol, pentobarbital, phenobarbital, secobarbital, amobarbital, delta-9-tetrahydrocannabinol (THC), combinations thereof, analogs thereof, and derivatives thereof.

7. A method of preventing drug use relapse due to cue inducement, including the steps of:
administering an effective amount of an α3β4 nicotinic antagonist to a mammal after an initial period of drug use; and
preventing a relapse of drug use during cue inducement.

8. The method of claim 7, wherein the α3β4 nicotinic antagonist is a coronaridine congener.

9. The method of claim 8, wherein the coronaridine congener is chosen from the group consisting of 18-hydroxycoronaridine; 18-hydroxyvoacangine; 18-hydroxyconopharyngine; 16-ethoxycarbonyl-18-hydroxyibogamine; 16-ethoxycarbonyl-18-hydroxyibogaine; 16-ethoxycarbonyl-18-hydroxyibogaline; 16-hydroxymethyl-18-hydroxyibogamine; 16-hydroxymethyl-18-hydroxyibogaine; 16-hydroxymethyl-18-hydroxyibogaline; 18-methoxycoronaridine; 18-methoxyvoacangine; 18-methoxyconopharyngine; 16-ethoxycarbonyl-18-methoxyibogamine; 16-ethoxycarbonyl-18-methoxyibogaine; 16-ethoxycarbonyl-18-methoxyibogaline; 16-hydroxymethyl-18-methoxyibogamine; 16-hydroxymethyl-18-methoxyibogaine; 16-hydroxymethyl-18-methoxyibogaline; 18-benzyloxycoronaridine; 18-benzyloxyvoacangine; 18-benzyloxyconopharyngine; 16-ethoxycarbonyl-18-benzyloxyibogamine; 16-ethoxycarbonyl-18-benzyloxyibogaine; 16-ethoxycarbonyl-18-benzyloxyibogaline; 18-hydroxycoronaridine laurate; 18-hydroxyvoacangine laurate; 18-hydroxyconopharyngine laurate; 16-ethoxycarbonyl-18-hydroxyibogamine laurate; 16-ethoxycarbonyl-18-hydroxyibogaine laurate; 16-ethoxycarbonyl-18-hydroxyibogaline laurate; 18-hydroxycoronaridine acetate; 18-hydroxyvoacangine acetate; 18-hydroxyconopharyngine acetate; 16-ethoxycarbonyl-18-hydroxyibogamine acetate; 16-ethoxycarbonyl-18-hydroxyibogaine acetate; 16-ethoxycarbonyl-18-hydroxyibogaline acetate; 18-hydroxycoronaridine methoxyethoxymethyl ether; 18-hydroxyvoacangine methoxyethoxymethyl ether; 18-hydroxyconopharyngine methoxyethoxymethyl ether; 16-ethoxycarbonyl-18-hydroxyibogamine methoxyethoxymethyl ether; 16-ethoxycarbonyl-18-hydroxyibogaine methoxyethoxymethyl ether; 16-ethoxycarbonyl-18-hydroxyibogaline methoxyethoxymethyl ether; and pharmaceutically acceptable salts thereof.

10. The method of claim 7, wherein the drug is chosen from the group consisting of a barbiturate, morphine, codeine, heroin, levorphanol, meperidine, methadone, propoxyphene, acetylmethadol (LAAM), pentazocine, butorphanol, nalbuphine, buprenorphine, dezocine, fentanyl, d-amphetamine, 1-amphetamine, d1-amphetamine, methamphetamine, 3,4-methylenedioxy-N-methylamphetamine (MDMA) benzphetamine, phentermine, diethylpropion, phenmetrazine, phendimetrazine, chlorphentermine, clortermine, mazindol, phenylpropanolamine, cocaine, methylphenidate, nicotine, cathinone (khat plant), meprobamate, chlordiazepoxide, diazepam, oxazepam, lorazepam, flurazepam, prazepam, chlorazepate, alprazolam, triazolam, temazepam, halazepam, quadazepam, midazolam, estazolam, ethanol, pentobarbital, phenobarbital, secobarbital, amobarbital, delta-9-tetrahydrocannabinol (THC), combinations thereof, analogs thereof, and derivatives thereof.

11. The method of claim 7, wherein the cue is chosen from the group consisting of music, drugs, drug paraphernalia, seeing others using drugs, environments where drugs were consumed, environments where drugs are supplied, arousal, anxiety, and discomfort.

12. The method of claim 7, wherein said administration step is further defined as orally delivering the α3β4 nicotinic antagonist.

13. The method of claim 7, wherein said administration step is further defined as administering 0.05 mg/kg to 200 mg/kg of the α3β4 nicotinic antagonist.

14. The method of claim 7, wherein said administering step is further defined as administering the α3β4 nicotinic antagonist at a time period after drug use.

15. The method of claim 7, wherein said preventing step further includes the step of reducing conditioned place preference (CPP) of the mammal.

16. The method of claim 7, further including the step of indirectly modulating the dopaminergic mesolimbic pathway via blockade of α3β4 nicotinic receptors in the habenulo-interpeduncular pathway and the basolateral amygdala.

17. A method of preventing drug use relapse due to cue inducement, including the steps of:
modulating the dopaminergic mesolimbic pathway by blocking α3β4 nicotinic receptors in the habenulo-interpeduncular pathway and the basolateral amygdala of a mammal after an initial period of drug use; and
preventing a relapse of drug use during cue inducement.

18. The method of claim 17, wherein the α3β4 nicotinic antagonist is a coronaridine congener chosen from the group consisting of 18-hydroxycoronaridine; 18-hydroxyvoacangine; 18-hydroxyconopharyngine; 16-ethoxycarbonyl-18-hydroxyibogamine; 16-ethoxycarbonyl-18-hydroxyibogaine; 16-ethoxycarbonyl-18-hydroxyibogaline; 16-hydroxymethyl-18-hydroxyibogamine; 16-hydroxymethyl-18-hydroxyibogaine; 16-hydroxymethyl-18-hydroxyibogaline; 18-methoxycoronaridine; 18-methoxyvoacangine; 18-methoxyconopharyngine; 16-ethoxycarbonyl-18-methoxyibogamine; 16-ethoxycarbonyl-18-methoxyibogaine; 16-ethoxycarbonyl-18-methoxyibogaline; 16-hydroxymethyl-18-methoxyibogamine; 16-hydroxymethyl-18-methoxyibogaine; 16-hydroxymethyl-18-methoxyibogaline; 18-benzyloxycoronaridine; 18-benzyloxyvoacangine; 18-benzyloxyconopharyngine; 16-ethoxycarbonyl-18-benzyloxyibogamine; 16-ethoxycarbonyl-18-benzyloxyibogaine; 16-ethoxycarbonyl-18-benzyloxyibogaline; 18-hydroxycoronaridine laurate; 18-hydroxyvoacangine laurate; 18-hydroxyconopharyngine laurate; 16-ethoxycarbonyl-18-hydroxyibogamine laurate; 16-ethoxycarbonyl-18-hydroxyibogaine laurate; 16-ethoxycarbonyl-18-hydroxyibogaline laurate; 18-hydroxycoronaridine acetate; 18-hydroxyvoacangine acetate; 18-hydroxyconopharyngine acetate; 16-ethoxycarbonyl-18-hydroxyibogamine acetate; 16-ethoxycarbonyl-18-hydroxyibogaine acetate; 16-ethoxycarbonyl-18-hydroxyibogaline acetate; 18-hydroxycoronaridine methoxyethoxymethyl ether; 18-hydroxyvoacangine methoxyethoxymethyl ether; 18-hydroxyconopharyngine methoxyethoxymethyl ether; 16-ethoxycarbonyl-18-hydroxyibogamine methoxyethoxymethyl ether; 16-ethoxycarbonyl-18-hydroxyibogaine methoxyethoxymethyl ether; 16-ethoxycarbonyl-18-hydroxyibogaline methoxyethoxymethyl ether; and pharmaceutically acceptable salts thereof.

19. A method of preventing drug use relapse, including the step of:
preventing a relapse of drug use during cue inducement.
